(19) United States
(12) Patent Application Publication
(10) Pub. No.: US 2023/0023070 A1
(43) Pub. Date: Jan. 26, 2023

(54) Therapy for the Treatment of Cancer

(71) Applicant: Celegene Corporation, Summit, NJ (US)

(72) Inventors: Chad Bjorklund, Summit, NJ (US); Hsiling Chiu, Livingston, NJ (US); Patrick Hagner, Sparta, NJ (US); Jian Kang, Warren, NJ (US); Anjan Thakurta, Basking Ridge, NJ (US)

(21) Appl. No.: 17/781,680

(22) PCT Filed: Dec. 1, 2020
(86) PCT No.: PCT/US20/62658

§ 371 Date: Jun. 1, 2022

Related U.S. Application Data
(60) Provisional application No. 62/942,378, filed on Dec. 2, 2019.

(51) Int. Cl. A61K 31/4035; A61K 38/55; A61P 35/00

(52) U.S. Cl. CPC A61K 31/4035; A61K 38/55; A61P 35/00

(57) ABSTRACT

Provided herein are methods of treating and/or managing cancer, which comprise administering to a patient Compound A, or an enantiomer or a mixture of enantiomers thereof, or a pharmaceutically acceptable salt, solvate, hydrate, co-crystal, clathrate, or polymorph thereof. Additionally, provided herein are methods of treating and/or managing cancer, which comprise administering to a patient Compound A, or an enantiomer or a mixture of enantiomers thereof, or a pharmaceutically acceptable salt, solvate, hydrate, co-crystal, clathrate, or polymorph thereof, in combination with a second agent selected from the group consisting of an anti-CD20 antibody, an HDAC inhibitor, a proteasome inhibitor, an anti-CD38 antibody, an anti-SLAMF7 antibody, a nuclear export inhibitor, a BCL-2 inhibitor, and an immune checkpoint inhibitor. Also provided herein are combination therapies for treating and/or managing cancer, which further comprise dexamethasone as a third agent.

This application claims priority to U.S. Provisional Application No. 62/942,378, filed on Dec. 2, 2019, the entirety of which is incorporated herein by reference.

### FIELD

Provided herein are therapies for treating and/or managing cancer, which comprise administering to a patient 3-(4-((4-(morpholinomethyl)benzyl)oxy)-1-oxoisoindolin-2-yl)piperidine-2,6-dione (“Compound A”), or an enantiomer or a mixture of enantiomers thereof, or a pharmaceutically acceptable salt, solvate, hydrate, co-crystal, clathrate, or polymorph thereof. Also provided are combination therapies for treating and/or managing cancer, which comprise administering to a patient 3-(4-((4-(morpholinomethyl)benzyl)oxy)-1-oxoisoindolin-2-yl)piperidine-2,6-dione (“Compound A”), or an enantiomer or a mixture of enantiomers thereof, or a pharmaceutically acceptable salt, solvate, hydrate, co-crystal, clathrate, or polymorph thereof, in combination with a second agent selected from the group consisting of an anti-CD20 antibody, an histone deacetylase (HDAC) inhibitor, a proteasome inhibitor, an anti-CD38 antibody, an anti-SLAMF7 antibody, a nuclear export inhibitor, a BCL-2 inhibitor, and an immune checkpoint inhibitor. Also provided herein are combination therapies for treating and/or managing cancer, which further comprise dexamethasone as a third agent.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

There is an enormous variety of cancers which are described in detail in the medical literature. Examples include cancer of the lung, colon, rectum, prostate, breast, brain, and intestine. The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

Many types of cancers are associated with new blood vessel formation, a process known as angiogenesis. Several of the mechanisms involved in tumor-induced angiogenesis have been elucidated. The most direct of these mechanisms is the secretion by the tumor cells of cytokines with angiogenic properties. Examples of these cytokines include acidic and basic fibroblastic growth factor (a,b-FGF), angiogenin, vascular endothelial growth factor (VEGF), and TNF-α. Alternatively, tumor cells can release angiogenic peptides through the production of proteases and the subsequent breakdown of the extracellular matrix where some cytokines are stored (e.g., b-FGF). Angiogenesis can also be induced indirectly through the recruitment of inflammatory cells (particularly macrophages) and their subsequent release of angiogenic cytokines (e.g., TNF-α, b-FGF).

Hematologic Cancers begin in blood-forming tissue, such as the bone marrow, or in the cells of the immune system. Examples of hematologic cancer are leukemia, lymphoma and multiple myeloma. Hematologic cancer is also called blood cancer.

Lymphoma refers to cancers that originate in the lymphatic system. Lymphoma is characterized by malignant neoplasms of lymphocytes—B lymphocytes and T lymphocytes (i.e., B-cells and T-cells). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body.

The treatment of various forms of lymphomas are described, for example, in U.S. Pat. No. 7,468,363, the entirety of which is incorporated herein by reference. Such lymphomas include, but are not limited to, Hodgkin's lymphoma, non-Hodgkin's lymphoma, cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma and mantle zone lymphoma and low grade follicular lymphoma.

Non-Hodgkin's lymphoma (NHL) is the fifth most common cancer for both men and women in the United States, with an estimated 63,190 new cases and 18,660 deaths in 2007. Jemal A, et al., CA Cancer J Clin 2007; 57(1):43-66. The probability of developing NHL increases with age and the incidence of NHL in the elderly has been steadily increasing in the past decade, causing concern with the aging trend of the US population. Id. Clarke C A, et al., Cancer 2002; 94(7):2015-2023. NHL is a cancer that starts in white blood cells. It is defined as being not Hodgkin lymphoma. NHL may be of B-cell, NK-cell or T-cell lymphoma. There are more than 60 subtypes of NHL, the most common are Diffuse Large B-cell Lymphoma (DLBCL), Follicular Lymphoma (FL), Mantle Cell Lymphoma (MCL), Small lymphocytic lymphoma, Double hit lymphoma, Primary mediastinal large B-cell Lymphoma, Splenic marginal zone B-cell lymphoma, Extranodal Marginal Zone B-cell lymphoma (MALT), Nodal Marginal Zone B-cell lymphoma and Lymphoplasmacytic lymphoma, Burkitt lymphoma, Primary Effusion Lymphoma are the most common B-cell lymphomas.

The most common T-cell lymphomas include Anaplastic large cell Lymphoma (systemic and cutaneous type), Peripheral T-Cell Lymphoma, Angioimmunoblastic T-cell lymphoma, Adult T-cell lymphoma/leukemia and Extranodal NK/T-cell lymphoma

Diffuse large B-cell lymphoma (DLBCL) accounts for approximately one-third of non-Hodgkin's lymphomas. While some DLBCL patients are cured with traditional chemotherapy, the remainder die from the disease. Anticancer drugs cause rapid and persistent depletion of lymphocytes, possibly by direct apoptosis induction in mature T and B cells. See K. Stahnke. et al., Blood 2001, 98:3066-3073. Absolute lymphocyte count (ALC) has been shown to be a prognostic factor in follicular non-Hodgkin's lymphoma and recent results have suggested that ALC at diagnosis is an important prognostic factor in diffuse large B-cell lymphoma. See D. Kim et al., Journal of Clinical Oncology, 2007 ASCO Annual Meeting Proceedings Part I. Vol 25, No. 18S (June 20 Supplement), 2007: 8082.

Leukemia refers to malignant neoplasms of the blood-forming tissues. Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17th ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17th ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or acute myeloblastic leukemia (AML), occurs at all ages and is the more common acute leukemia among adults; it is the form usually associated with irradiation as a causative agent.

Chronic leukemias are described as being lymphocytic (CLL) or myelocytic (CIVIL). The Merck Manual, 949-952 (17th ed. 1999). CLL is characterized by the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CML is relatively easy to diagnose because of the presence of the Philadelphia chromosome.

In addition to the acute and chronic categorization, neoplasms are also categorized based upon the cells giving rise to such disorder into precursor or peripheral. See e.g., U.S. patent publication no. 2008/0051379, which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, diffuse large B-cell lymphoma and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin. M-protein, short for monoclonal protein, also known as paraprotein, is a particularly abnormal protein produced by the myeloma plasma cells and can be found in the blood or urine of almost all patients with multiple myeloma.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

The incidence of cancer continues to climb as the general population ages, as new cancers develop, and as susceptible populations (e.g., people infected with AIDS, the elderly or excessively exposed to sunlight) grow. A tremendous demand therefore exists for new methods, treatments and compositions that can be used to treat patients with cancer including but not limited to those with lymphoma, NHL, multiple myeloma, AML, leukemias, and solid tumors.

Non-Hodgkin lymphoma (NHL) represents a wide spectrum of neoplasms derived from normal lymphoid cells. WHO lymphoma classification is utilized to define subtypes based on clinical, pathological, phenotypical and molecular features (Swerdlow, 2016). In North America and Europe, 85-90% of NHL are derived from B-cells while 10-15% from T-cells (Laurent, 2018). NK lymphomas are very rare.

NHL is the most frequent hematological malignancy and it is estimated that, in 2019 in the US, 74.200 new cases of NHL will be diagnosed, and that 19.970 people will die from this disease (Siegel 2019).

Classical Hodgkin lymphoma (cHL) is a B-cell neoplasm that most often involves cervical and mediastinal regions. It accounts for 95% of all cases of Hodgkin lymphoma (HL). Epidemiology is characterized by a bimodal age curve with a peak in the third decade and a second after 60 years. It is estimated that, in 2019 in the US, 8.110 new cases of HL will be diagnosed, and that 1000 people will die from this disease (Siegel 2019).

Most patients present at diagnosis with unique of more often multiple superficial or profound lymphadenopathies, with or without systemic symptoms. However, since lymphoma can involve any organ, many other presentations are possible. Initial workup typically includes precise diagnosis according to current WHO classification, analysis of disease extension and evaluation of comorbidities. Prognosis and initial management are subtype-specific (Armitage, 2017).

There exists a significant need for safe and effective methods of treating, preventing and managing cancer, particularly for cancers that are refractory to standard treatments, such as surgery, radiation therapy, chemotherapy and hormonal therapy, while reducing or avoiding the toxicities and/or side effects associated with the conventional therapies.

### SUMMARY

Provided herein are methods of treating or managing cancer. In one aspect, provided herein is a method of treating or managing cancer, comprising administering to a patient in need of such treatment or management an amount of from about 0.01 mg to about 5 mg per day of a compound, wherein the compound is Compound A

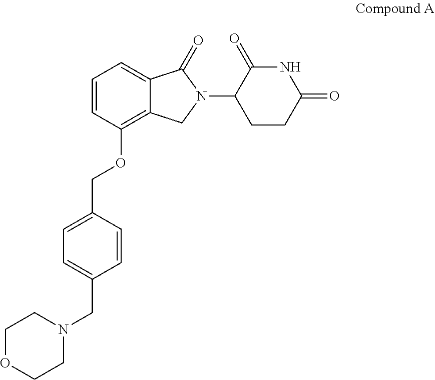 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In another aspect, provided herein is a method of treating or managing cancer, comprising administering to a patient in need of such treatment or management (i) a therapeutically effective amount of a compound in combination with (ii) an anti-CD20 antibody, wherein the compound is Compound A

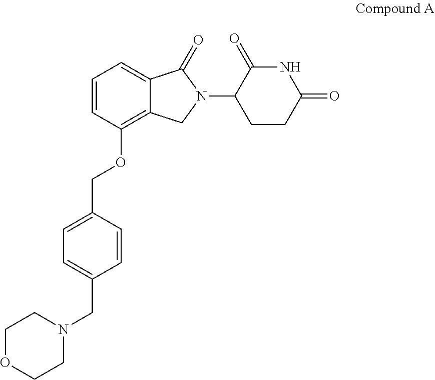 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the anti-CD20 antibody is obinutuzumab. In another embodiment, the anti-CD20 antibody is rituximab.

In another aspect, provided herein is a method of treating or managing cancer, comprising administering to a patient in need of such treatment or management (i) a therapeutically effective amount of a compound in combination with (ii) an HDAC inhibitor, wherein the compound is Compound A

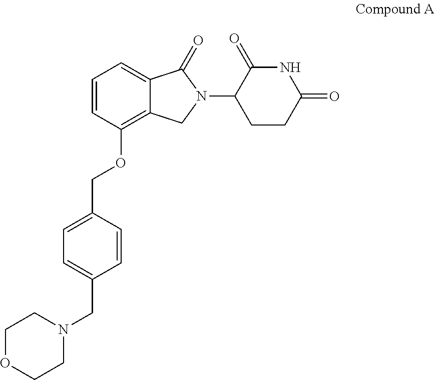 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the HDAC inhibitor is citarinostat (ACY-241), or a pharmaceutically acceptable salt, solvate, hydrate, co-crystal, clathrate, or polymorph thereof.

In another aspect, provided herein is a method of treating or managing cancer, comprising administering to a patient in need of such treatment or management (i) a therapeutically effective amount of a compound in combination with (ii) a proteasome inhibitor, wherein the compound is Compound A

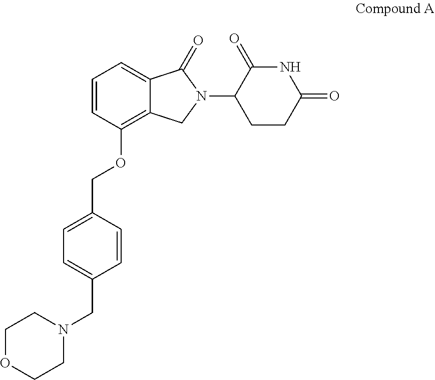 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the proteasome inhibitor is marizomib (salinosporamide A), or an enantiomer or a mixture of enantiomers thereof, or a pharmaceutically acceptable salt, solvate, hydrate, co-crystal, clathrate, or polymorph thereof.

In one embodiment, the proteasome inhibitor is bortezomib, or an enantiomer or a mixture of enantiomers thereof, or a pharmaceutically acceptable salt, solvate, hydrate, co-crystal, clathrate, or polymorph thereof.

In one embodiment, the proteasome inhibitor is carfilzomib, or an enantiomer or a mixture of enantiomers thereof, or a pharmaceutically acceptable salt, solvate, hydrate, co-crystal, clathrate, or polymorph thereof.

In one embodiment, the proteasome inhibitor is ixazomib, or an enantiomer or a mixture of enantiomers thereof, or a pharmaceutically acceptable salt, solvate, hydrate, co-crystal, clathrate, or polymorph thereof.

In another aspect, provided herein is a method of treating or managing cancer, comprising administering to a patient in need of such treatment or management (i) a therapeutically effective amount of a compound in combination with (ii) an anti-CD38 antibody, wherein the compound is Compound A

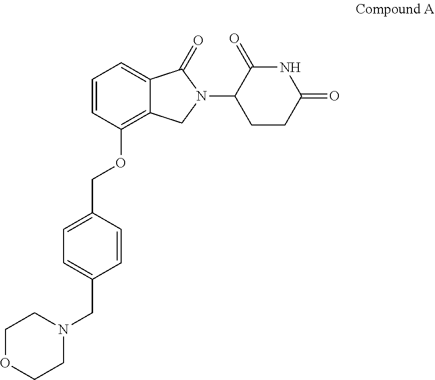 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the anti-CD38 antibody is isatuximab.

In one embodiment, the anti-CD38 antibody is daratumumab.

In another aspect, provided herein is a method of treating or managing cancer, comprising administering to a patient in need of such treatment or management (i) a therapeutically effective amount of a compound in combination with (ii) an anti-SLAMF7 antibody, wherein the compound is Compound A

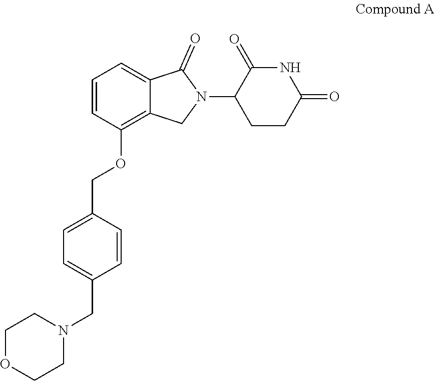 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the anti-SLAMF7 antibody is elotuzumab.

In another aspect, provided herein is a method of treating or managing cancer, comprising administering to a patient in need of such treatment or management (i) a therapeutically effective amount of a compound in combination with (ii) a nuclear export inhibitor, wherein the compound is Compound A

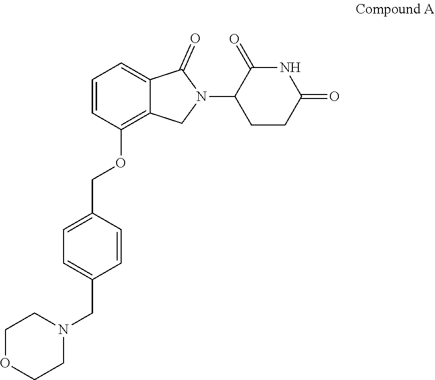 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the nuclear export inhibitor is selinexor, or a geometric isomer or a mixture of geometric isomers thereof, or a pharmaceutically acceptable salt, solvate, hydrate, co-crystal, clathrate, or polymorph thereof.

In yet another aspect, provided herein is a method of treating or managing cancer, comprising administering to a patient in need of such treatment or management (i) a therapeutically effective amount of a compound in combination with (ii) a BCL-2 inhibitor, wherein the compound is Compound A

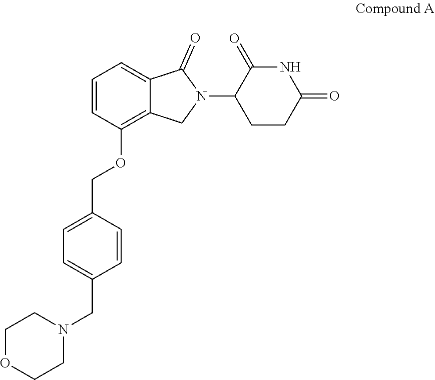 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the BCL-2 inhibitor is venetoclax, or a pharmaceutically acceptable salt, solvate, hydrate, co-crystal, clathrate, or polymorph thereof.

In yet another aspect, provided herein is a method of treating or managing cancer, comprising administering to a patient in need of such treatment or management (i) a therapeutically effective amount of a compound in combination with (ii) an immune checkpoint inhibitor, wherein the compound is Compound A

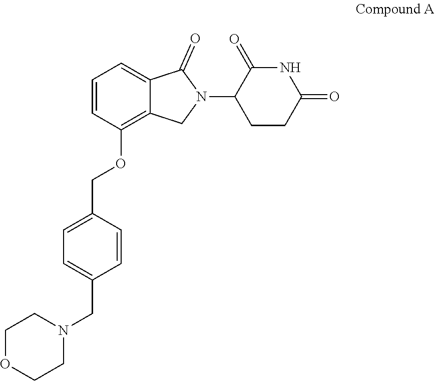 [SMILES: O=C1CCC(N2Cc3c(OCc4ccc(CN5CCOCC5)cc4)cccc3C2=O)C(=O)N1]

or an enantiomer or mixture of enantiomers thereof, or a pharmaceutically acceptable salt, solvate, hydrate, co-crystal, clathrate, or polymorph thereof.

In one embodiment, the immune checkpoint inhibitor is pembrolizumab.

In one embodiment, the immune checkpoint inhibitor is nivolumab.

In one embodiment, the immune checkpoint inhibitor is ipilimumab.

In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof.

In certain embodiments, the cancer is relapsed or refractory. In certain embodiments, the cancer is newly diagnosed.

In certain embodiments, the cancer is non-Hodgkin lymphoma (NHL). In certain embodiments, the NHL is diffuse large B-cell lymphoma (DLBCL), follicular lymphoma (FL), marginal zone lymphoma (MZL), mantle cell lymphoma (MCL), peripheral T-cell lymphoma (PTCL), or primary central nervous system lymphoma (PCNSL).

In certain embodiments, the NHL is relapsed or refractory NHL.

In certain embodiments, the cancer is Hodgkin Lymphoma (HL). In certain embodiments, the HL is classical Hodgkin Lymphoma (cHL).

In certain embodiments, the HL is relapsed or refractory HL.

### BRIEF DESCRIPTION OF THE FIGURES

FIG. 1 illustrates the synergistic anti-proliferative activity of Compound A-S in combination with bortezomib. PI: proteasome inhibitor; POM: pomalidomide; BORT: bortezomib; CI: combination index.

FIG. 2 illustrates apoptosis induced by Compound A-S in combination with proteasome inhibitors, bortezomib or carfilzomib. POM: pomalidomide; BORT: bortezomib; CFZ: carfilzomib.

### DETAILED DESCRIPTION

### Definitions

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise specified, the term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of the compounds provided herein, with or without other additional active agent, after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of the compounds provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the disease remains in remission.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

Combination therapy or “in combination with” refer to the use of more than one therapeutic agent to treat a particular disorder or condition. By “in combination with,” it is not intended to imply that the therapeutic agents must be administered at the same time and/or formulated for delivery together, although these methods of delivery are within the scope of this application. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

As used herein, the terms “additional active agent,” “active agent” and “active ingredient” refer to pharmacologically active compounds useful in the treatment of particular types of cancer, and certain diseases and conditions associated with or characterized by undesired angiogenesis. The active agents can be large molecules (e.g., proteins) or small molecules (e.g., synthetic inorganic, organometallic, or organic molecules). Examples of large molecule active agents include, but are not limited to, hematopoietic growth factors, cytokines, and monoclonal and polyclonal antibodies. In certain embodiments, large molecule active agents are biological molecules, such as naturally occurring or artificially made proteins. Proteins that are particularly useful in this application include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; GC-CSF, BCG, cancer antibodies, and EPO. Active agents that are small molecules can also be used to alleviate adverse effects associated with the administration of the compounds provided herein. However, like some large molecules, many are believed to be capable of providing a synergistic effect when administered with (e.g., before, after or simultaneously) the compounds provided herein. Examples of small molecule additional active agents include, but are not limited to, anti-cancer agents, antibiotics, immunosuppressive agents, and steroids.

In certain embodiments, the active agent is at least one chemotherapeutic agent, at least one anti-inflammatory agent, or at least one immunosuppressive and/or immunomodulatory agent. In one embodiment, such a chemotherapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine, cladribine and similar agents. In one embodiment, such a chemotherapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin, and similar agents. In one embodiment, such a chemotherapeutic agent may be selected from an antibiotic, such as dactinomycin (formerly actinomycin), bleomycin, daunorubicin (formerly daunomycin), idarubicin, mithramycin, mitomycin, mitoxantrone, plicamycin, anthramycin (AMC) and similar agents. In one embodiment, such a chemotherapeutic agent may be selected from an anti-mitotic agent, such as taxanes, for instance docetaxel, and paclitaxel. In one embodiment, such a chemotherapeutic agent may be selected from a topoisomerase inhibitor, such as topotecan. In one embodiment, such a chemotherapeutic agent may be selected from a growth factor inhibitor, such as an inhibitor of ErbB1 (EGFR) (such as gefitinib (Iressa®), cetuximab (Erbitux®), erlotinib (Tarceva®), 2F8 (provided in WO 2002/100348) and similar agents), an inhibitor of ErbB2 (Her2/neu) (such as trastuzumab (Herceptin®) and similar agents) and similar agents. In one embodiment, such a growth factor inhibitor may be a farnesyl transferase inhibitor, such as SCH-66336 and R115777. In one, embodiment, such a growth factor inhibitor may be a vascular endothelial growth factor (VEGF) inhibitor, such as bevacizumab (Avastin®). In one embodiment, such a chemotherapeutic agent may be a tyrosine kinase inhibitor, such as imatinib (Glivec, Gleevec STI571), lapatinib, PTK787/ZK222584 and similar agents. In one embodiment, such a chemotherapeutic agent may be a histone deacetylase inhibitor. Examples of such histone deacetylase inhibitors include hydroxamic acid-based hybrid polar compounds, such as SAHA (suberoylanilide hydroxamic acid). In one embodiment, such a chemotherapeutic agent may be a P38a MAP kinase inhibitor, such as SCIO-469.

In a further embodiment, the therapy of the invention further includes administration of at least one inhibitor of angiogenesis, neovascularization, and/or other vascularization to a subject in need thereof. Examples of such angiogenesis inhibitors are urokinase inhibitors, matrix metalloprotease inhibitors (such as marimastat, neovastat, BAY 12-9566, AG 3340, BMS-275291 and similar agents), inhibitors of endothelial cell migration and proliferation (such as TNP-470, squalamine, 2-methoxyestradiol, combretastatins, endostatin, angiostatin, penicillamine, SCH66336 (Schering-Plough Corp, Madison, N.J.), R115777 (Janssen Pharmaceutica, Inc, Titusville, N.J.) and similar agents), antagonists of angiogenic growth factors (such as such as ZD6474, SU6668, antibodies against angiogenic agents and/or their receptors (such as VEGF, bFGF, and angiopoietin-1), Sugen 5416, SU5402, antiangiogenic ribozyme (such as angiozyme), interferon α (such as interferon α2a), suramin and similar agents), VEGF-R kinase inhibitors and other anti-angiogenic tyrosine kinase inhibitors (such as SU011248), inhibitors of endothelial-specific integrin/survival signaling (such as vitaxin and similar agents), copper antagonists/chelators (such as tetrathiomolybdate, captopril and similar agents), carboxyamido-triazole (CAI), ABT-627, CM101, interleukin-12 (IL-12), IM862, PNU145156E as well as nucleotide molecules inhibiting angiogenesis (such as antisense-VEGF-cDNA, cDNA coding for angiostatin, cDNA coding for p53 and cDNA coding for deficient VEGF receptor-2) and similar agents. Other examples of such inhibitors of angiogenesis, neovascularization, and/or other vascularization are anti-angiogenic heparin derivatives and related molecules (e.g., heperinase III), temozolomide, NK4, macrophage migration inhibitory factor (MIF), cyclooxygenase-2 inhibitors, inhibitors of hypoxia-inducible factor 1, anti-angiogenic soy isoflavones, oltipraz, fumagillin and analogs thereof, somatostatin analogues, pentosan polysulfate, tecogalan sodium, dalteparin, tumstatin, thrombospondin, NM-3, combrestatin, canstatin, avastatin, antibodies against other relevant targets (such as anti-alpha-v/beta-3 integrin and anti-kininostatin mAbs) and similar agents.

In a further embodiment, the therapy of the invention further includes administration of an anti-cancer immunogen, such as a cancer antigen/tumor-associated antigen (e.g., epithelial cell adhesion molecule (EpCAM/TACSTD1), mucin 1 (MUC1), carcinoembryonic antigen (CEA), tumor-associated glycoprotein 72 (TAG-72), gp100, Melan-A, MART-1, KDR, RCAS1, MDA7, cancer-associated viral vaccines (e.g., human papillomavirus vaccines), tumor-derived heat shock proteins, and similar agents. A number of other suitable cancer antigens/tumor-associated antigens described elsewhere herein and similar molecules known in the art may also or alternatively be used in such embodiment. Anti-cancer immunogenic peptides also include anti-idiotypic “vaccines” such as BEC2 anti-idiotypic antibodies, Mitumomab, CeaVac and related anti-idiotypic antibodies, anti-idiotypic antibody to MG7 antibody, and other anti-cancer anti-idiotypic antibodies (see for instance Birebent et al., Vaccine. 21(15), 1601-12 (2003), Li et al., Chin Med J (Engl). 114(9), 962-6 (2001), Schmitt et al., Hybridoma. 13(5), 389-96 (1994), Maloney et al., Hybridoma. 4(3), 191-209 (1985), Raychardhuri et al., J Immunol. 137(5), 1743-9 (1986), Pohl et al., Int J Cancer. 50(6), 958-67 (1992), Bohlen et al., Cytokines Mol Ther. 2(4), 231-8 (1996) and Maruyama, J Immunol Methods. 264(1-2), 121-33 (2002)). Such anti-idiotypic Abs may optionally be conjugated to a carrier, which may be a synthetic (typically inert) molecule carrier, a protein (for instance keyhole limpet hemocyanin (KLH) (see for instance Ochi et al., Eur J Immunol. 17(11), 1645-8 (1987)), or a cell (for instance a red blood cell—see for instance Wi et al., J Immunol Methods. 122(2), 227-34 (1989)). In a further embodiment, the therapy of the invention further includes administration of a bisphosphonate. Examples of potentially suitable biphosphonates are pamidronate (Aredia®), zoledronic acid (Zometa®), clodronate (Bonefos®), risendronate (Actonel®), ibandronate (Boniva®), etidronate (Didronel®), alendronate (Fosamax®), tiludronate (Skelid®), incadronate (Yamanouchi Pharmaceutical) and minodronate (YM529, Yamanouchi). In a further embodiment, the therapy of the invention further includes administration of a colony stimulating factor. Examples of suitable colony stimulating factors are granulocyte-colony stimulating factors (G-CSF), such as filgrastim (Neupogen®) and pegfilgrastim (Neulasta®), and granulocyte macrophage-colony stimulating factors (GM-CSF) such as sargramostim (Leukine®). In a further embodiment, the therapy of the invention further includes administration of an erythropoietic agent. Examples of suitable erythropoietic agents are erythropoietin (EPO), such as epoetin alfa (for instance Procrit®, Epogen®, and Eprex®) and epoetin beta (for instance NeoRecormon®) and erythropoiesis-stimulating proteins (for instance Aranesp®). In a further embodiment, the therapy of the invention further includes administration of an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNα (e.g., INFα2b), IFNβ, GM-CSF, CD40L, Flt3 ligand, stem cell factor, ancestim, and TNFα. Suitable chemokines may include Glu-Leu-Arg (ELR)-negative chemokines such as IP-10, MCP-3, MIG, and SDF-1α from the human CXC and C-C chemokine families. Suitable cytokines include cytokine derivatives, cytokine variants, cytokine fragments, and cytokine fusion proteins. In a further embodiment, the therapy of the invention further includes administration of an agent that modulates, e.g., enhances or inhibits, the expression or activity of Fcα or Fcγ receptors. Examples of agents suitable for this use include interleukin-1 (IL-1), interleukin-2 (IL-2), interleukin-6 (IL-6), granulocyte colony-stimulating factor (G-CSF), such as filgrastim (Neupogen®) and pegfilgrastim (Neulasta®), and granulocyte macrophage-colony stimulating factors (GM-CSF) such as sargramostim (Leukine®), interferon-γ (IFN-γ), and tumor necrosis factor (TNF). In a further embodiment, the therapy of the invention further includes administration of a cell cycle control/apoptosis regulator (or “regulating agent”). A cell cycle control/apoptosis regulator may include molecules (i) that target and modulate cell cycle control/apoptosis regulators such as cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), agents inducing NF-κB blockade leading to inhibition of IL-6 production, antibodies that activate TRAIL receptors, IFNs, anti-sense Bcl-2, and As2O3 (arsenic trioxide, Trisenox®). In a further embodiment, the therapy of the invention further includes administration of a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxyprogesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane), a hormone inhibitor (such as octreotide/-sandostatin) and similar agents. In a further embodiment, the therapy of the invention further includes administration of an anti-anergic agent (for instance small molecule compounds, proteins, glycoproteins, or antibodies that break tolerance to tumor and cancer antigens). Examples of such compounds are molecules that block the activity of CTLA-4, such as MDX-010 (Phan et al., PNAS USA 100, 8372 (2003)). In a further embodiment, the therapy of the invention further includes administration of a tumor suppressor gene-containing nucleic acid or vector such as a replication-deficient adenovirus encoding human recombinant wild-type p53/SCH58500, etc.; antisense nucleic acids targeted to oncogenes, mutated, or deregulated genes; or siRNA targeted to mutated or deregulated genes. Examples of tumor suppressor targets include, for example, BRCA1, RB1, BRCA2, DPC4 (Smad4), MSH2, MLH1, and DCC.

In a further embodiment, the therapy of the invention further includes administration of an anti-cancer nucleic acid, such as genasense (augmerosen/G3139), LY900003 (ISIS 3521), ISIS 2503, OGX-011 (ISIS 112989), LE-AON/LEraf-AON (liposome encapsulated c-raf antisense oligonucleotide/ISIS-5132), MG98, and other antisense nucleic acids that target PKCα, clusterin, IGFBPs, protein kinase A, cyclin D1, or Bcl-2h. In a further embodiment, the therapy of the invention further includes administration of an anti-cancer inhibitory RNA molecule (see for instance Lin et al., Curr Cancer Drug Targets. 1(3), 241-7 (2001), Erratum in: Curr Cancer Drug Targets. 3(3), 237 (2003), Lima et al., Cancer Gene Ther. 11(5), 309-16 (2004), Grzmil et al., Int J Oncol. 4(1), 97-105 (2004), Collis et al., Int J Radiat Oncol Biol Phys. 57(2 Suppl), S144 (2003), Yang et al., Oncogene. 22(36), 5694-701 (2003) and Zhang et al., Biochem Biophys Res Commun. 303(4), 1169-78 (2003)). In a further embodiment, the therapy of the invention further includes administration of a virus, viral proteins, and the like. Replication-deficient viruses, that generally are capable of one or only a few rounds of replication in vivo, and that are targeted to tumor cells, may for instance be useful components of such compositions and methods. Such viral agents may comprise or be associated with nucleic acids encoding immunostimulants, such as GM-CSF and/or IL-2. Both naturally oncolytic and such recombinant oncolytic viruses (for instance HSV-1 viruses, reoviruses, replication-deficient and replication-sensitive adenovirus, etc.) may be useful components of such methods and compositions (see for instance Shah et al., J Neurooncol. 65(3), 203-26 (2003), Stiles et al., Surgery. 134(2), 357-64 (2003), Sunarmura et al., Pancreas. 28(3), 326-9 (2004), Teshigahara et al., J Surg Oncol. 85(1), 42-7 (2004), Varghese et al., Cancer Gene Ther. 9(12), 967-78 (2002), Wildner et al., Cancer Res. 59(2), 410-3 (1999), Yamanaka, Int J Oncol. 24(4), 919-23 (2004) and Zwiebel et al., Semin Oncol. 28(4), 336-43 (2001). In a further embodiment, the therapy of the invention may further involve “whole cell” and “adoptive” immunotherapy methods. For instance, such methods may comprise infusion or re-infusion of immune system cells (for instance tumor-infiltrating lymphocytes (TILs), such as CD4+ and/or CD8+ T cells (for instance T cells expanded with tumor-specific antigens and/or genetic enhancements), antibody-expressing B cells or other antibody producing/presenting cells, dendritic cells (e.g., anti-cytokine expressing recombinant dendritic cells, dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887, July 2001), optionally admixed with adjuvants such as alum, may also be components in such methods and combination compositions. In a further embodiment, the therapy of the invention further includes the application of an internal vaccination method. Internal vaccination refers to induced tumor or cancer cell death, such as drug-induced or radiation-induced cell death of tumor cells, in a patient, that typically leads to elicitation of an immune response directed towards (i) the tumor cells as a whole or (ii) parts of the tumor cells including (a) secreted proteins, glycoproteins or other products, (b) membrane-associated proteins or glycoproteins or other components associated with or inserted in membranes, and/or (c) intracellular proteins or other intracellular components. An internal vaccination-induced immune response may be humoral (i.e. antibody—complement-mediated) or cell-mediated (e.g., the development and/or increase of endogenous cytotoxic T lymphocytes that recognize the internally killed tumor cells or parts thereof). In a further embodiment, the therapy of the invention further includes administration of complement. Accordingly, the use of compositions comprising anti-CD38 antibodies with serum or complement is also within the scope of the present invention. In these compositions the complement is located in close proximity to the anti-CD38 antibody, for instance by conjugation or may be suited for simultaneous administration. Alternatively, the anti-CD38 antibodies and the complement or serum may be administered separately. In a further embodiment, the therapy of the invention further includes administration of differentiation inducing agents, retinoic acid and retinoic acid analogues (such as all trans retinoic acid, 13-cis retinoic acid and similar agents), vitamin D analogues (such as seocalcitol and similar agents), inhibitors of ErbB3, ErbB4, IGF-IR, insulin receptor, PDGFRa, PDGFRbeta, Flk2, Flt4, FGFR1, FGFR2, FGFR3, FGFR4, TRKA, TRKC, c-met, Ron, Sea, Tie, Tie2, Eph, Ret, Ros, Alk, LTK, PTK7 and similar agents. In a further embodiment, the therapy of the invention further includes administration of a cathepsin B, modulators of cathepsin D dehydrogenase activity, glutathione-S-transferase (such as glutacylcysteine synthetase and lactate dehydrogenase), or similar agents. In a further embodiment, the therapy of the invention further includes administration of estramustine or epirubicin. In a further embodiment, the therapy of the invention further includes administration of a HSP90 inhibitor like 17-allyl amino geld-anamycin, antibodies directed against a tumor antigen such as PSA, CA125, KSA, etc., integrins like integrin (31, inhibitors of VCAM or similar agents

In a further embodiment, the therapy of the invention further includes administration of calcineurin-inhibitors (such as valspodar, PSC 833 and other MDR-1 or p-glycoprotein inhibitors), TOR-inhibitors (such as sirolimus, everolimus and rapamycin). and inhibitors of “lymphocyte homing” mechanisms (such as FTY720), and agents with effects on cell signaling such as adhesion molecule inhibitors (for instance anti-LFA, etc.). In a further embodiment, the therapy of the invention further includes radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT), brachytherapy (BT) or skeletal targeted radiotherapy). Radioactive elements that may be used in practicing such methods include, e.g., radium, cesium-137, iridium-192, americium-241, gold-198, cobalt-57, copper-67, technetium-99, iodide-123, iodide-131, and indium-111. In a further embodiment, the therapy of the invention further includes autologous peripheral stem cell or bone marrow transplantation. In a further embodiment, the therapy of the invention further includes orthopedic intervention. Orthopedic interventions may be used in the treatment of a disorder involving cells expressing CD38, such as multiple myeloma, to help control pain or retain function or mobility. Such interventions may include physical therapy, splinting of bones to prevent or treat fractures, or surgical procedures (minor or major) to repair fractures. In a further embodiment, the therapy of the invention further includes delivery of one or more agents that promote access of the CD38 antibody or combination composition to the interior of a tumor. Such methods may for example be performed in association with the delivery of a relaxin, which is capable of relaxing a tumor (see for instance U.S. Pat. No. 6,719,977). In one embodiment, the anti-CD38 antibody used in the present invention may be bonded to a cell penetrating peptide (CPP). Cell penetrating peptides and related peptides (such as engineered cell penetrating antibodies) are described in for instance Zhao et al., J Immunol Methods. 254(1-2), 137-45 (2001), Hong et al., Cancer Res. 60(23), 6551-6 (2000). Lindgren et al., Biochem J. 377(Pt 1), 69-76 (2004), Buerger et al., J Cancer Res Clin Oncol. 129(12), 669-75 (2003), Pooga et al., FASEB J. 12(1), 67-77 (1998) and Tseng et al., Mol Pharmacol. 62(4), 864-72 (2002).

In a further embodiment, the therapy of the invention further includes administration of at least one anti-inflammatory agent. In one embodiment such an anti-inflammatory agent may be selected from a steroidal drug and a NSAID (nonsteroidal anti-inflammatory drug). In one embodiment such an anti-inflammatory agent may be selected from aspirin and other salicylates, Cox-2 inhibitors (such as rofecoxib and celecoxib), NSAIDs (such as ibuprofen, fenoprofen, naproxen, sulindac, diclofenac, piroxicam, ketoprofen, diflunisal, nabumetone, etodolac, oxaprozin, and indomethacin), anti-IL6R antibodies, anti-IL8 antibodies (e.g. 10F8 described in WO2004/058797), anti-IL15 antibodies, anti-IL15R antibodies, anti-CD4 antibodies, anti-CD11a antibodies (e.g., efalizumab), anti-alpha-4/beta-1 integrin (VLA4) antibodies (e.g natalizumab), CTLA4-Ig for the treatment of inflammatory diseases, prednisolone, prednisone, disease modifying antirheumatic drugs (DMARDs) such as methotrexate, hydroxychloroquine, sulfasalazine, pyrimidine synthesis inhibitors (such as leflunomide), IL-1 receptor blocking agents (such as anakinra), TNF-α blocking agents (such as etanercept, infliximab, and adalimumab) and similar agents.

In a further embodiment, the therapy of the invention further includes administration of at least one immunosuppressive and/or immunomodulatory agent to a subject in need thereof. In one embodiment, such an immunosuppressive and/or immunomodulatory agent may be selected from cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In one embodiment, such an immunosuppressive and/or immunomodulatory agent may be selected from immunosuppressive antibodies, such as antibodies binding to p75 of the IL-2 receptor, or antibodies binding to for instance MHC, CD2, CD3, CD4, CD7, CD28, B7, CD40, CD45, IFNγ, TNF-α, IL-4, IL-5, IL-6R, IL-6; IGF, IGFR1, IL-7, IL-8, IL-10, CD11a, or CD58, or antibodies binding to their ligands. In one embodiment, such an immunosuppressive and/or immunomodulatory agent may be selected from soluble IL-15R, IL-10, B7 molecules (B7-1, B7-2, variants thereof, and fragments thereof), ICOS, and OX40, an inhibitor of a negative T cell regulator (such as an antibody against CTLA4) and similar agents. In a further embodiment, the therapy of the invention further includes administration of an anti-C3b(i) antibody.

In a further embodiment, the therapy of the invention further includes administration of histone deacetylase inhibitors (for instance phenylbutyrate) and/or DNA repair agents (for instance DNA repair enzymes and related compositions such as dimericine). In a further embodiment, the therapy of the invention further includes anti-cancer directed photodynamic therapy (for instance anti-cancer laser therapy-which optionally may be practiced with the use of photosensitizing agent, see, for instance Zhang et al., J Control Release. 93(2), 141-50 (2003)), anti-cancer sound-wave and shock-wave therapies (see for instance Kambe et al., Hum Cell. 10(1), 87-94 (1997)), and/or anti-cancer nutraceutical therapy (see for instance Roudebush et al., Vet Clin North Am Small Anim Pract. 34(1), 249-69, viii (2004) and Rafi, Nutrition. 20(1), 78-82 (2004).

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

As used herein, and unless otherwise specified, the term “tumor” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

As used herein, and unless otherwise specified, the term “relapsed” refers to a situation where a subject or a mammal, which has had a remission of cancer after therapy has a return of cancer cells.

As used herein, and unless otherwise specified, an “effective patient tumor response” refers to any increase in the therapeutic benefit to the patient. An “effective patient tumor response” can be, for example, a 5%, 10%, 25%, 50%, or 100% decrease in the rate of progress of the tumor. An “effective patient tumor response” can be, for example, a 5%, 10%, 25%, 50%, or 100% decrease in the physical symptoms of a cancer. An “effective patient tumor response” can also be, for example, a 5%, 10%, 25%, 50%, 100%, 200%, or more increase in the response of the patient, as measured by any suitable means, such as gene expression, cell counts, assay results, etc.

As used herein, and unless otherwise specified, the term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

As used herein, and unless otherwise specified, the term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

As used herein, and unless otherwise specified, the term “monitor,” as used herein, generally refers to the overseeing, supervision, regulation, watching, tracking, or surveillance of an activity. For example, the term “monitoring the effectiveness of a compound” refers to tracking the effectiveness in treating a cancer in a patient or in a tumor cell culture. Similarly, the “monitoring,” when used in connection with patient compliance, either individually, or in a clinical trial, refers to the tracking or confirming that the patient is actually taking the immunomodulatory compound being tested as prescribed. The monitoring can be performed, for example, by following the expression of mRNA or protein biomarkers.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

As used herein, and unless otherwise specified, the term “refractory or resistant” refers to a circumstance where a subject or a mammal, even after intensive treatment, has residual cancer cells in his body.

As used herein, and unless otherwise specified, the term “drug resistance” refers to the condition when a disease does not respond to the treatment of a drug or drugs. Drug resistance can be either intrinsic, which means the disease has never been responsive to the drug or drugs, or it can be acquired, which means the disease ceases responding to a drug or drugs that the disease had previously responded to. In certain embodiments, drug resistance is intrinsic. In certain embodiments, the drug resistance is acquired.

As used herein, and unless otherwise specified, the term “sensitivity” and “sensitive” when made in reference to treatment with compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase of, at least a 5%, or more, in the effectiveness of the tumor treatment.

As used herein, and unless otherwise specified, the terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

As used herein and unless otherwise specified, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. In certain embodiments, a stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, greater than about 70% by weight, or greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### Clinical Trial Endpoints for Cancer Approval

“Overall survival” (OS) is defined as the time from first dose until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval.

Several endpoints are based on cancer assessments. These endpoints include disease free survival (DFS), objective response rate (ORR), time to progression (TTP), progression-free survival (PFS), event-free survival (EFS), duration of response (DOR) and time-to-treatment failure (TTF). The collection and analysis of data on these time-dependent endpoints are based on indirect assessments, calculations, and estimates.

Generally, “disease free survival” (DFS) is defined as the time from randomization until recurrence of cancer or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior cancer progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

“Objective response rate” (ORR) is defined as the sum of the percentage of patients who achieve complete and partial responses. Response duration usually is measured from the time of initial response until documented cancer progression. Generally, the FDA has defined ORR as the sum of partial responses plus complete responses. When defined in this manner, ORR is a direct measure of drug anticancer activity, which can be evaluated in a single-arm study. If available, standardized criteria should be used to ascertain response. A variety of response criteria have been considered appropriate (e.g., RECIST criteria) (Therasse et al., (2000) J. Natl. Cancer Inst, 92: 205-16). The significance of ORR is assessed by its magnitude and duration, and the percentage of complete responses (no detectable evidence of cancer).

“Duration of response” (DOR) is the time from achieving a response until relapse or disease progression.

“Time to progression” (TTP) and “progression-free survival” (PFS) have served as primary endpoints for drug approval. TTP is defined as the time from randomization until objective cancer progression; TTP does not include deaths. PFS is defined as the time from randomization until objective cancer progression or death. Compared with TTP, PFS is the preferred regulatory endpoint. PFS includes deaths and thus can be a better correlate to overall survival. PFS assumes patient deaths are randomly related to cancer progression. However, in situations where the majority of deaths are unrelated to cancer, TTP can be an acceptable endpoint.

As an endpoint to support drug approval, PFS can reflect cancer growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

“Event-free survival” (EFS) is the time from study entry until any treatment failure, including disease progression, treatment discontinuation for any reason, or death.

“Time-to-treatment failure” (TTF) is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient or physician withdrawal, or patient intolerance.

In certain embodiments, the methods provided herein are useful for achieving one or more of these clinical trial endpoints in a patient. In certain embodiments, the methods provided herein are useful for improving one or more of these clinical trial endpoints in a patient.

### Compounds

In certain embodiments, the compound for use in the compositions and methods provided herein is 3-(4-((4-(morpholinomethyl)benzyl)oxy)-1-oxoisoindolin-2-yl)piperidine-2,6-dione (Compound A), having the following structure:

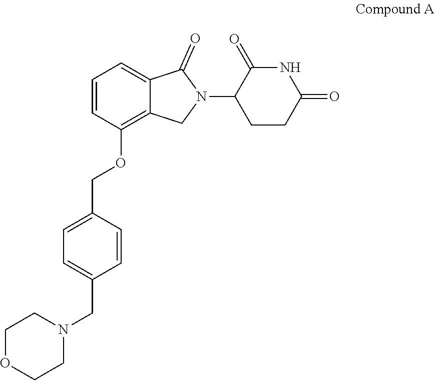 [SMILES: O=C1CCC(N2Cc3c(OCc4ccc(CN5CCOCC5)cc4)cccc3C2=O)C(=O)N1]

or an enantiomer or a mixture of enantiomers thereof; or a pharmaceutically acceptable salt, solvate, hydrate, co-crystal, clathrate, or polymorph thereof.

In one embodiment, the compound is 3-(4-((4-(morpholinomethyl)benzyl)oxy)-1-oxoisoindolin-2-yl)piperidine-2,6-dione. In one embodiment, the compound is a pharmaceutically acceptable salt of Compound A. In one embodiment, the compound is 3-(4-((4-(morpholinomethyl)benzyl)oxy)-1-oxoisoindolin-2-yl)piperidine-2,6-dione hydrochloride.

In one embodiment, the compound is (S)-3-(4-((4-(morpholinomethyl)benzyl)oxy)-1-oxoisoindolin-2-yl)piperidine-2,6-dione (Compound A-S), having the following structure:

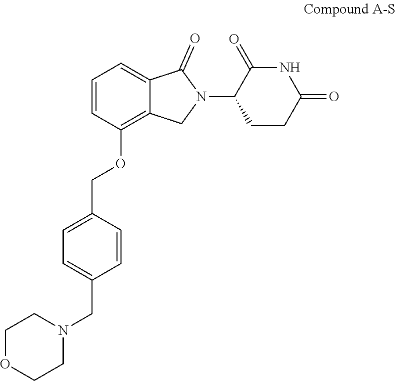 [SMILES: O=C1CC[C@H](N2Cc3c(OCc4ccc(CN5CCOCC5)cc4)cccc3C2=O)C(=O)N1]

In one embodiment, the compound is a pharmaceutically acceptable salt of Compound A-S. In one embodiment, the compound is a hydrochloride salt of Compound A-S.

In one embodiment, the compound is (R)-3-(4-((4-(morpholinomethyl)benzyl)oxy)-1-oxoisoindolin-2-yl)piperidine-2,6-dione (Compound A-R), having the following structure:

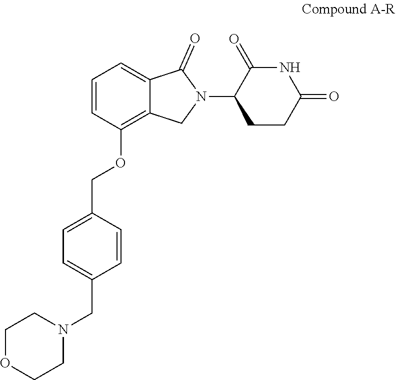 [SMILES: O=C1CC[C@@H](N2Cc3c(OCc4ccc(CN5CCOCC5)cc4)cccc3C2=O)C(=O)N1]

In one embodiment, the compound is a pharmaceutically acceptable salt of compound A-R. In one embodiment, the compound is (R)-3-(4-((4-(morpholinomethyl)benzyl)oxy)-1-oxoisoindolin-2-yl)piperidine-2,6-dione hydrochloride.

Compound A can be prepared according to the methods described in U.S. Application Publication Nos. US2011-0196150 and US2014-0045843, the entirety of each of which is incorporated herein by reference. The compound can be also synthesized according to other methods apparent to those of skill in the art based upon the teaching of these publications.

Compounds provided herein markedly inhibit TNF-α, IL-10, and other inflammatory cytokines in LPS-stimulated hPBMC and human whole blood. TNF-α is an inflammatory cytokine produced by macrophages and monocytes during acute inflammation. TNF-α is responsible for a diverse range of signaling events within cells. TNF-α may play a pathological role in cancer. Without being limited by theory, one of the biological effects exerted by the immunomodulatory compounds provided herein is the reduction of synthesis of TNF-α. The immunomodulatory compounds provided herein enhance the degradation of TNF-α mRNA. The compounds provided herein also potently inhibit IL-1 β and stimulates IL-10 under these conditions.

Further, without being limited by any particular theory, the compounds provided herein are potent co-stimulators of T cells and increase cell proliferation in a dose dependent manner under appropriate conditions.

In certain embodiments, without being limited by theory, the biological effects exerted by the immunomodulatory compounds provided herein include, but not limited to, anti-angiogenic and immune modulating effects.

Compound A provided herein contains one chiral center, and can exist as a mixture of enantiomers, e.g., a racemic mixture. This application encompasses the use of stereomerically pure forms of such a compound, as well as the use of mixtures of those forms. For example, mixtures comprising equal or unequal amounts of the enantiomers of Compound A provided herein may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

In certain embodiments, the compound for use in the compositions and methods provided herein is a monoclonal antibody that binds to CD20. In certain embodiments, the anti-CD20 antibody is obinutuzumab. In certain embodiments, the anti-CD20 antibody is rituximab.

In certain embodiments, the compound for use in the compositions and methods provided herein is an HDAC inhibitor. In certain embodiments, the HDAC inhibitor is 2-(N-(2-chlorophenyl)anilino)-N-[7-(hydroxyamino)-7-oxoheptyl]pyrimidine-5-carboxamide, having the following structure:

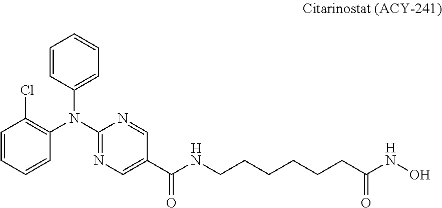

or a pharmaceutically acceptable salt, solvate, hydrate, co-crystal, clathrate, or polymorph thereof. In certain embodiments, the HDAC inhibitor is citarinostat (ACY-241).

In certain embodiments, the compound for use in the compositions and methods provided herein is a proteasome inhibitor.

In certain embodiments, the proteasome inhibitor is (1R,4R,5S)-4-(2-chloroethyl)-1-[(S)-[(1S)-cyclohex-2-en-1-yl]-hydroxymethyl]-5-methyl-6-oxa-2-azabicyclo[3.2.0]heptane-3,7-dione, having the following structure:

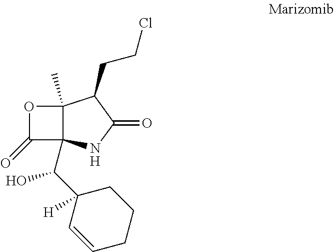

or an enantiomer or a mixture of enantiomers thereof, or a pharmaceutically acceptable salt, solvate, hydrate, co-crystal, clathrate, or polymorph thereof. In certain embodiments, the proteasome inhibitor is marizomib.

In certain embodiments, the proteasome inhibitor is [(1R)-3-methyl-1-[[(2S)-3-phenyl-2-(pyrazine-2-carbonylamino)propanoyl]amino]butyl]boronic acid, having the following structure:

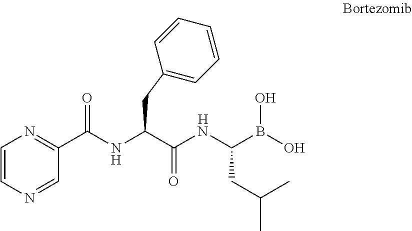

or an enantiomer or a mixture of enantiomers thereof, or a pharmaceutically acceptable salt, solvate, hydrate, co-crystal, clathrate, or polymorph thereof. In certain embodiments, the proteasome inhibitor is bortezomib.

In certain embodiments, the proteasome inhibitor is (2S)-4-methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide, having the following structure:

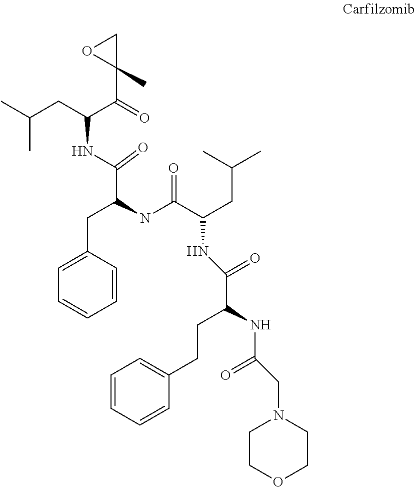

or an enantiomer or a mixture of enantiomers thereof, or a pharmaceutically acceptable salt, solvate, hydrate, co-crystal, clathrate, or polymorph thereof. In certain embodiments, the proteasome inhibitor is carfilzomib.

In certain embodiments, the proteasome inhibitor is [(1R)-1-[[2-[(2,5-dichlorobenzoyl)amino]acetyl]amino]-3-methylbutyl]boronic acid, having the following structure:

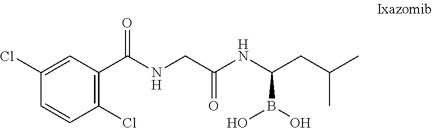

or an enantiomer or a mixture of enantiomers thereof, or a pharmaceutically acceptable salt, solvate, hydrate, co-crystal, clathrate, or polymorph thereof. In certain embodiments, the proteasome inhibitor is ixazomib.

In certain embodiments, the compound for use in the compositions and methods provided herein is a monoclonal antibody that binds to CD38. In certain embodiments, the anti-CD38 antibody is isatuximab. In certain embodiments, the anti-CD38 antibody is daratumumab.

In certain embodiments, the compound for use in the compositions and methods provided herein is a monoclonal antibody that binds to SLAMF7. In certain embodiments, the anti-SLAMF7 antibody is elotuzumab.

In certain embodiments, the compound for use in the compositions and methods provided herein is a nuclear export inhibitor. In certain embodiments, the nuclear export inhibitor is (Z)-3-[3-[3,5-bis(trifluoromethyl)phenyl]-1,2,4-triazol-1-yl]-N′-pyrazin-2-ylprop-2-enehydrazide, having the following structure:

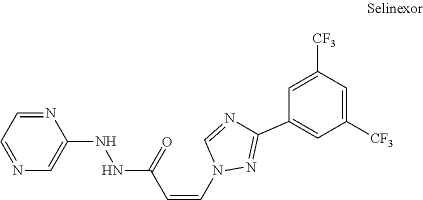 [SMILES: O=C(/C=C\n1cnc(-c2cc(C(F)(F)F)cc(C(F)(F)F)c2)n1)NNc1cnccn1]

or a geometric isomer or a mixture of geometric isomers thereof, or a pharmaceutically acceptable salt, solvate, hydrate, co-crystal, clathrate, or polymorph thereof. In certain embodiments, the nuclear export inhibitor is selinexor.

In certain embodiments, the compound for use in the compositions and methods provided herein is a BCL-2 inhibitor. In certain embodiments, the BCL-2 inhibitor is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide, having the following structure:

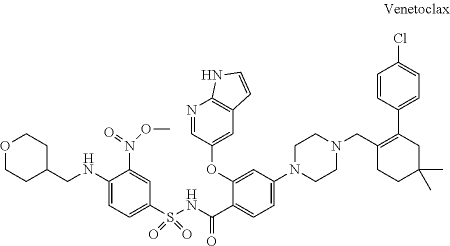

or a pharmaceutically acceptable salt, solvate, hydrate, co-crystal, clathrate, or polymorph thereof. In certain embodiments, the BCL-2 inhibitor is venetoclax, or a pharmaceutically acceptable salt, solvate, hydrate, co-crystal, clathrate, or polymorph thereof.

In certain embodiments, the compound for use in the compositions and methods provided herein is a monoclonal antibody that inhibits an immune checkpoint. In certain embodiments, the immune checkpoint inhibitor is pembrolizumab. In certain embodiments, the immune checkpoint inhibitor is nivolumab. In certain embodiments, the immune checkpoint inhibitor is ipilimumab.

In certain embodiments, the compound for use in the compositions and methods provided herein is (11b,16a)-9-fluoro-11,17,21-trihydroxy-16-methylpregna-1,4-diene-3,20-dione, having the following structure:

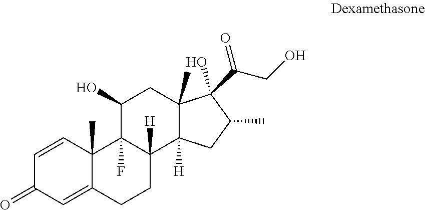

or an enantiomer or a mixture of enantiomers thereof, or a pharmaceutically acceptable salt, solvate, hydrate, co-crystal, clathrate, or polymorph thereof.

In one embodiment, the compound is (11b,16a)-9-fluoro-11,17,21-trihydroxy-16-methylpregna-1,4-diene-3,20-dione. In one embodiment, the compound is a pharmaceutically acceptable salt of dexamethasone. In one embodiment, the compound is dexamethasone sodium phosphate.

Dexamethasone can be prepared according to the methods described in U.S. Pat. Nos. 2,990,401 and 3,035,050, the entirety of each of which is incorporated herein by reference.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### Methods of Treatment and Compounds for Use in Such Methods

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods of treating and/or managing cancer. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof, as a part of a combination therapy. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods of treating and/or managing cancer. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with an anti-CD20 antibody. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the anti-CD20 antibody is obinutuzumab. In certain embodiments, the anti-CD20 antibody is rituximab.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with an HDAC inhibitor. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the HDAC inhibitor is citarinostat, or a pharmaceutically acceptable salt, solvate, hydrate, co-crystal, clathrate, or polymorph thereof.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with a proteasome inhibitor. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the proteasome inhibitor is marizomib, or an enantiomer or a mixture of enantiomers thereof, or a pharmaceutically acceptable salt, solvate, hydrate, co-crystal, clathrate, or polymorph thereof. In certain embodiments, the proteasome inhibitor is bortezomib, or an enantiomer or a mixture of enantiomers thereof, or a pharmaceutically acceptable salt, solvate, hydrate, co-crystal, clathrate, or polymorph thereof. In certain embodiments, the proteasome inhibitor is carfilzomib, or an enantiomer or a mixture of enantiomers thereof, or a pharmaceutically acceptable salt, solvate, hydrate, co-crystal, clathrate, or polymorph thereof. In certain embodiments, the proteasome inhibitor is ixazomib, or an enantiomer or a mixture of enantiomers thereof, or a pharmaceutically acceptable salt, solvate, hydrate, co-crystal, clathrate, or polymorph thereof.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with an anti-CD38 antibody. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the anti-CD38 antibody is isatuximab. In certain embodiments, the anti-CD38 antibody is daratumumab.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with an anti-SLAMF7 antibody. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the anti-SLAMF7 antibody is elotuzumab.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with a nuclear export inhibitor. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the nuclear export inhibitor is selinexor, or a geometric isomer or a mixture of geometric isomers thereof, or a pharmaceutically acceptable salt, solvate, hydrate, co-crystal, clathrate, or polymorph thereof.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with a BCL-2 inhibitor. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the BCL-2 inhibitor is venetoclax, or a pharmaceutically acceptable salt, solvate, hydrate, co-crystal, clathrate, or polymorph thereof.

Provided herein are methods of treating and/or managing cancer, which comprise administering to a patient in need of such treatment and/or management a therapeutically or prophylactically effective amount of 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof in combination with an immune checkpoint inhibitor. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof. Provided herein is 3-(4-((4-(morpholinomethyl)benzyl)oxy)-1-oxoisoindolin-2-yl)piperidine-2,6-dione (Compound A), or an enantiomer or a mixture of enantiomers thereof, or a pharmaceutically acceptable salt, solvate, hydrate, co-crystal, clathrate, or polymorph thereof for use in such methods. In some embodiments, the compound is (S)-3-(4-((4-(morpholinomethyl)benzyl)oxy)-1-oxoisoindolin-2-yl)piperidine-2,6-dione (Compound A-S) or a pharmaceutically acceptable salt, solvate, hydrate, co-crystal, clathrate, or polymorph thereof. In some embodiments, the compound is hydrochloride salt of Compound A-S. In some embodiments, the compound is (R)-3-(4-((4-(morpholinomethyl)benzyl)oxy)-1-oxoisoindolin-2-yl)piperidine-2,6-dione (Compound A-R) or a pharmaceutically acceptable salt, solvate, hydrate, co-crystal, clathrate, or polymorph thereof. In certain embodiments, the immune checkpoint inhibitor is pembrolizumab. In certain embodiments, the immune checkpoint inhibitor is nivolumab. In certain embodiments, the immune checkpoint inhibitor is ipilimumab.

As used herein, the term “cancer” includes, but is not limited to, blood born tumors. In certain embodiments, term “cancer” includes karotype acute myeloblastic leukemia, multiple myeloma, Hodgkin's lymphoma, non-Hodgkin's lymphoma, cutaneous T-Cell lymphoma, cutaneous B-Cell lymphoma, diffuse large B-Cell lymphoma and low grade follicular lymphoma.

In certain embodiments, the cancer is a hematological tumor. In certain embodiments, the hematological tumor is metastatic. In certain embodiments, the hematological tumor is drug resistant. In certain embodiments, the cancer is myeloma or lymphoma.

In certain embodiments, the myeloma is multiple myeloma. In certain embodiments, the multiple myeloma is smoldering myeloma, indolent myeloma, active multiple myeloma, extramedullary plasmacytoma, solitary plasmacytoma of the bone, light chain myeloma, or non-secretory myeloma. In certain embodiments, the multiple myeloma is relapsed, refractory or resistant multiple myeloma. In certain embodiments, the multiple myeloma is relapsed and refractory multiple myeloma.

Provided herein are methods of treating or managing myeloma, particularly multiple myeloma. In some embodiments, provided herein are methods for the treatment or management of smoldering myeloma, indolent myeloma, active multiple myeloma, extramedullary plasmacytoma, solitary plasmacytoma of the bone, light chain myeloma, or non-secretory myeloma. In some embodiments, provided herein are methods for the treatment or management of relapsed, refractory or resistant multiple myeloma. In some embodiments, provided herein are methods for the treatment or management of relapsed and refractory multiple myeloma.

In certain embodiments, the lymphoma is Hodgkin's lymphoma, classical Hodgkin's lymphoma (cHL), non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, follicular lymphoma (FL), marginal zone lymphoma (MZL), transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, primary central nervous system lymphoma (PCNSL), or low grade follicular lymphoma.

In certain embodiments, the lymphoma is NHL. In certain embodiments, the NHL is diffuse large B-cell lymphoma (DLBCL), follicular lymphoma (FL), marginal zone lymphoma (MZL), mantle cell lymphoma (MCL), peripheral T-cell lymphoma (PTCL), or primary central nervous system lymphoma (PCNSL). In certain embodiments, the NHL is DLBCL. In certain embodiments, the NHL is FL. In certain embodiments, the NHL is MZL. In certain embodiments, the NHL is MCL. In certain embodiments, the NHL is PTCL. In certain embodiments, the NHL is PCNSL.

In certain embodiments, the NHL is relapsed or refractory NHL. In certain embodiments, the NHL is relapsed or refractory DLBCL. In certain embodiments, the NHL is relapsed or refractory FL. In certain embodiments, the NHL is relapsed or refractory MZL. In certain embodiments, the NHL is relapsed or refractory MCL. In certain embodiments, the NHL is relapsed or refractory PTCL. In certain embodiments, the NHL is relapsed or refractory PCNSL. In certain embodiments, the subject has failed at least one prior therapy.

In certain embodiments, the NHL is newly diagnosed.

In certain embodiments, the lymphoma is Hodgkin Lymphoma (HL).

In certain embodiments, the HL is classical Hodgkin Lymphoma (cHL).

In certain embodiments, the HL is relapsed or refractory HL.

In certain embodiments, the HL is relapsed or refractory cHL.

In certain embodiments, the HL is newly diagnosed.

In certain embodiments, provided herein are methods for the treatment or management of Hodgkin's lymphoma (HL), classical Hodgkin's lymphoma (cHL), non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, follicular lymphoma (FL), marginal zone lymphoma (MZL), transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, primary central nervous system lymphoma (PCNSL), or low grade follicular lymphoma.

In certain embodiments, provided herein are methods of treating or managing NHL. In some embodiments, provided herein are methods for the treatment or management of DLBCL, FL, MZL, MCL, PTCL, or PCNSL. In certain embodiments, provided herein are methods of treating or managing HL. In certain embodiments, provided herein are methods of treating or managing cHL.

In certain embodiments, provided herein are methods for the treatment or management of relapsed or refractory cancer. In certain embodiments, provided herein are methods for the treatment or management of relapsed or refractory Hodgkin's lymphoma (HL), relapsed or refractory classical Hodgkin's lymphoma (cHL), relapsed or refractory non-relapsed or refractory Hodgkin's lymphoma (NHL), relapsed or refractory cutaneous B-cell lymphoma, activated B-cell lymphoma, relapsed or refractory diffuse large B-cell lymphoma (DLBCL), relapsed or refractory mantle cell lymphoma (MCL), relapsed or refractory follicular center lymphoma, relapsed or refractory follicular lymphoma (FL), relapsed or refractory marginal zone lymphoma (MZL), relapsed or refractory transformed lymphoma, relapsed or refractory lymphocytic lymphoma of intermediate differentiation, relapsed or refractory intermediate lymphocytic lymphoma (ILL), relapsed or refractory diffuse poorly differentiated lymphocytic lymphoma (PDL), relapsed or refractory centrocytic lymphoma, relapsed or refractory diffuse small-cleaved cell lymphoma (DSCCL), relapsed or refractory peripheral T-cell lymphomas (PTCL), relapsed or refractory cutaneous T-Cell lymphoma, relapsed or refractory primary central nervous system lymphoma (PCNSL), or relapsed or refractory low grade follicular lymphoma.

In certain embodiments, provided herein are methods of treating or managing relapsed or refractory NHL. In some embodiments, provided herein are methods for the treatment or management of relapsed or refractory DLBCL, relapsed or refractory FL, relapsed or refractory MZL, relapsed or refractory MCL, relapsed or refractory PTCL, or relapsed or refractory PCNSL. In certain embodiments, provided herein are methods of treating or managing relapsed or refractory HL. In certain embodiments, provided herein are methods of treating or managing relapsed or refractory cHL.

In certain embodiments, provided herein are methods of treating or managing newly diagnosed NHL. In some embodiments, provided herein are methods for the treatment or management of newly diagnosed DLBCL, newly diagnosed FL, newly diagnosed MZL, newly diagnosed MCL, newly diagnosed PTCL, or newly diagnosed PCNSL. In certain embodiments, provided herein are methods of treating or managing newly diagnosed HL. In certain embodiments, provided herein are methods of treating or managing newly diagnosed cHL.

Provided herein are methods of treating cancer, e.g., NHL and HL, which result in an improvement in overall survival of the patient. In some embodiments, the improvement in overall survival of the patient is observed in a patient population sensitive to treatment with Compound A, or an enantiomer or a mixture of enantiomers thereof, or a pharmaceutically acceptable salt, solvate, hydrate, co-crystal, clathrate, or polymorph thereof. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-CD20 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD20 antibody is obinutuzumab. In certain embodiments, the anti-CD20 antibody is rituximab.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an HDAC inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the HDAC inhibitor is citarinostat (ACY-241), or a pharmaceutically acceptable salt, solvate, hydrate, co-crystal, clathrate, or polymorph thereof.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a proteasome inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the proteasome inhibitor is marizomib, or an enantiomer or a mixture of enantiomers thereof, or a pharmaceutically acceptable salt, solvate, hydrate, co-crystal, clathrate, or polymorph thereof. In certain embodiments, the proteasome inhibitor is bortezomib, or an enantiomer or a mixture of enantiomers thereof, or a pharmaceutically acceptable salt, solvate, hydrate, co-crystal, clathrate, or polymorph thereof. In certain embodiments, the proteasome inhibitor is carfilzomib, or an enantiomer or a mixture of enantiomers thereof, or a pharmaceutically acceptable salt, solvate, hydrate, co-crystal, clathrate, or polymorph thereof. In certain embodiments, the proteasome inhibitor is ixazomib, or an enantiomer or a mixture of enantiomers thereof, or a pharmaceutically acceptable salt, solvate, hydrate, co-crystal, clathrate, or polymorph thereof.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-CD38 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD38 antibody is isatuximab. In certain embodiments, the anti-CD38 antibody is daratumumab.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-SLAMF7 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-SLAMF7 antibody is elotuzumab.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a nuclear export inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the nuclear export inhibitor is selinexor, or a geometric isomer or a mixture of geometric isomers thereof, or a pharmaceutically acceptable salt, solvate, hydrate, co-crystal, clathrate, or polymorph thereof.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a BCL-2 inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the BCL-2 inhibitor is venetoclax, or a pharmaceutically acceptable salt, solvate, hydrate, co-crystal, clathrate, or polymorph thereof.

Also provided herein are methods of treating cancer, e.g., NHL and HL, which result in an improvement in overall survival of the patient. In some embodiments, the improvement in overall survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an immune checkpoint inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the immune checkpoint inhibitor is pembrolizumab. In certain embodiments, the immune checkpoint inhibitor is nivolumab. In certain embodiments, the immune checkpoint inhibitor is ipilimumab.

Provided herein are methods of treating cancer, e.g., NHL and HL, which result in an improvement in disease free survival of the patient. In some embodiments, disease free survival of the patient is observed in a patient population sensitive to treatment with Compound A, or an enantiomer or a mixture of enantiomers thereof, or a pharmaceutically acceptable salt, solvate, hydrate, co-crystal, clathrate, or polymorph thereof. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-CD20 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD20 antibody is obinutuzumab. In certain embodiments, the anti-CD20 antibody is rituximab.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an HDAC inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the HDAC inhibitor is citarinostat, or a pharmaceutically acceptable salt, solvate, hydrate, co-crystal, clathrate, or polymorph thereof.

Also provided herein are methods of treating cancer, e.g., NHL and HL, which result in an improvement in disease free survival of the patient. In some embodiments, disease free survival of the patient t is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a proteasome inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the proteasome inhibitor is marizomib, or an enantiomer or a mixture of enantiomers thereof, or a pharmaceutically acceptable salt, solvate, hydrate, co-crystal, clathrate, or polymorph thereof. In certain embodiments, the proteasome inhibitor is bortezomib, or an enantiomer or a mixture of enantiomers thereof, or a pharmaceutically acceptable salt, solvate, hydrate, co-crystal, clathrate, or polymorph thereof. In certain embodiments, the proteasome inhibitor is carfilzomib, or an enantiomer or a mixture of enantiomers thereof, or a pharmaceutically acceptable salt, solvate, hydrate, co-crystal, clathrate, or polymorph thereof. In certain embodiments, the proteasome inhibitor is ixazomib, or an enantiomer or a mixture of enantiomers thereof, or a pharmaceutically acceptable salt, solvate, hydrate, co-crystal, clathrate, or polymorph thereof.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-CD38 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD38 antibody is isatuximab. In certain embodiments, the anti-CD38 antibody is daratumumab.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-SLAMF7 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-SLAMF7 antibody is elotuzumab.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a nuclear export inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the nuclear export inhibitor is selinexor, or a geometric isomer or a mixture of geometric isomers thereof, or a pharmaceutically acceptable salt, solvate, hydrate, co-crystal, clathrate, or polymorph thereof.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a BCL-2 inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the BCL-2 inhibitor is venetoclax, or a pharmaceutically acceptable salt, solvate, hydrate, co-crystal, clathrate, or polymorph thereof.

Also provided herein are methods of treating cancer, e.g., NHL and HL, which result in an improvement in disease free survival of the patient. In some embodiments, disease free survival of the patient is observed in a patient population sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an immune checkpoint inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the immune checkpoint inhibitor is pembrolizumab. In certain embodiments, the immune checkpoint inhibitor is nivolumab. In certain embodiments, the immune checkpoint inhibitor is ipilimumab.

Provided herein are methods of treating cancer, e.g., NHL and HL, which result in an improvement in the objective response rate in the patient population. In some embodiments, the patient population is sensitive to treatment with Compound A, or an enantiomer or a mixture of enantiomers thereof, or a pharmaceutically acceptable salt, solvate, hydrate, co-crystal, clathrate, or polymorph thereof. In certain embodiments, the methods further comprise administering dexamethasone, or an enantiomer or a mixture of enantiomers thereof, or a pharmaceutically acceptable salt, solvate, hydrate, co-crystal, clathrate, or polymorph thereof.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-CD20 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD20 antibody is obinutuzumab. In certain embodiments, the anti-CD20 antibody is rituximab.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an HDAC inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the HDAC inhibitor is citarinostat, or a pharmaceutically acceptable salt, solvate, hydrate, co-crystal, clathrate, or polymorph thereof.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a proteasome inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the proteasome inhibitor is marizomib, or an enantiomer or a mixture of enantiomers thereof, or a pharmaceutically acceptable salt, solvate, hydrate, co-crystal, clathrate, or polymorph thereof. In certain embodiments, the proteasome inhibitor is bortezomib, or an enantiomer or a mixture of enantiomers thereof, or a pharmaceutically acceptable salt, solvate, hydrate, co-crystal, clathrate, or polymorph thereof. In certain embodiments, the proteasome inhibitor is carfilzomib, or an enantiomer or a mixture of enantiomers thereof, or a pharmaceutically acceptable salt, solvate, hydrate, co-crystal, clathrate, or polymorph thereof. In certain embodiments, the proteasome inhibitor is ixazomib, or an enantiomer or a mixture of enantiomers thereof, or a pharmaceutically acceptable salt, solvate, hydrate, co-crystal, clathrate, or polymorph thereof.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-CD38 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD38 antibody is isatuximab. In certain embodiments, the anti-CD38 antibody is daratumumab.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an anti-SLAMF7 antibody. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-SLAMF7 antibody is elotuzumab.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a nuclear export inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the nuclear export inhibitor is selinexor, or a geometric isomer or a mixture of geometric isomers thereof, or a pharmaceutically acceptable salt, solvate, hydrate, co-crystal, clathrate, or polymorph thereof.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) a BCL-2 inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the BCL-2 inhibitor is venetoclax, or a pharmaceutically acceptable salt, solvate, hydrate, co-crystal, clathrate, or polymorph thereof.

Also provided herein are methods of treating cancer, e.g., NHL and HL, which result in an improvement in the objective response rate in the patient population. In some embodiments, the patient population is sensitive to treatment with (i) Compound A, or an enantiomer or a mixture of enantiomers thereof, or a pharmaceutically acceptable salt, solvate, hydrate, co-crystal, clathrate, or polymorph thereof in combination with (ii) an immune checkpoint inhibitor. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the immune checkpoint inhibitor is pembrolizumab. In certain embodiments, the immune checkpoint inhibitor is nivolumab. In certain embodiments, the immune checkpoint inhibitor is ipilimumab.

In some embodiments, Compound A, or an enantiomer or a mixture of enantiomers thereof, or a pharmaceutically acceptable salt, solvate, hydrate, co-crystal, clathrate, or polymorph thereof, is administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with an anti-CD20 antibody, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with obinutuzumab, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with rituximab, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with an HDAC inhibitor, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with citarinostat, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with a proteasome inhibitor, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with marizomib, or an enantiomer or a mixture of enantiomers thereof, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with bortezomib, or an enantiomer or a mixture of enantiomers thereof, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with carfilzomib, or an enantiomer or a mixture of enantiomers thereof, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with ixazomib, or an enantiomer or a mixture of enantiomers thereof, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with an anti-CD38 antibody, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with isatuximab, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with daratumumab, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with an anti-SLAMF7 antibody, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with elotuzumab, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with a nuclear export inhibitor, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with selinexor, or a geometric isomer or a mixture of geometric isomers thereof, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof

In some embodiments, Compound A, or an enantiomer or a mixture of enantiomers thereof, or a pharmaceutically acceptable salt, solvate, hydrate, co-crystal, clathrate, or polymorph thereof, in combination with a BCL-2 inhibitor, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with venetoclax, or a pharmaceutically acceptable salt, solvate, hydrate, co-crystal, clathrate, or polymorph thereof, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, in combination with an immune checkpoint inhibitor, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with pembrolizumab, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with nivolumab, are administered in combination with dexamethasone, or an enantiomer or a mixture of enantiomers thereof, or a pharmaceutically acceptable salt, solvate, hydrate, co-crystal, clathrate, or polymorph thereof. In some embodiments, Compound A, or an enantiomer or a mixture of enantiomers thereof, or a pharmaceutically acceptable salt, solvate, hydrate, co-crystal, clathrate, or polymorph thereof, in combination with ipilimumab, are administered in combination with dexamethasone, or an enantiomer or a mixture of enantiomers thereof, or a pharmaceutically acceptable salt, solvate, hydrate, co-crystal, clathrate, or polymorph thereof.

In some embodiments, Compound A, or an enantiomer or a mixture of enantiomers thereof, or a pharmaceutically acceptable salt, solvate, hydrate, co-crystal, clathrate, or polymorph thereof, is administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with an anti-CD20 antibody, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with obinutuzumab, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with rituximab, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with an HDAC inhibitor, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with citarinostat, or a pharmaceutically acceptable salt, solvate, hydrate, co-crystal, clathrate, or polymorph thereof, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with a proteasome inhibitor, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with marizomib, or an enantiomer or a mixture of enantiomers thereof, or a pharmaceutically acceptable salt, solvate, hydrate, co-crystal, clathrate, or polymorph thereof, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with bortezomib, or an enantiomer or a mixture of enantiomers thereof, or a pharmaceutically acceptable salt, solvate, hydrate, co-crystal, clathrate, or polymorph thereof, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with carfilzomib, or an enantiomer or a mixture of enantiomers thereof, or a pharmaceutically acceptable salt, solvate, hydrate, co-crystal, clathrate, or polymorph thereof, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with ixazomib, or an enantiomer or a mixture of enantiomers thereof, or a pharmaceutically acceptable salt, solvate, hydrate, co-crystal, clathrate, or polymorph thereof, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with an anti-CD38 antibody, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with isatuximab, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with daratumumab, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with an anti-SLAMF7 antibody, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with elotuzumab, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with a nuclear export inhibitor, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with selinexor, or a geometric isomer or a mixture of geometric isomers thereof, or a pharmaceutically acceptable salt, solvate, hydrate, co-crystal, clathrate, or polymorph thereof, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with a BCL-2 inhibitor, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with venetoclax, or a pharmaceutically acceptable salt, solvate, hydrate, co-crystal, clathrate, or polymorph thereof, are administered in combination with a therapy conventionally used to treat or manage cancer.

In some embodiments, Compound A, or an enantiomer or a mixture of enantiomers thereof, or a pharmaceutically acceptable salt, solvate, hydrate, co-crystal, clathrate, or polymorph thereof, in combination with an immune checkpoint inhibitor, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with pembrolizumab, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with nivolumab, are administered in combination with a therapy conventionally used to treat or manage cancer. In some embodiments, Compound A, or an enantiomer or a mixture of enantiomers thereof, or a pharmaceutically acceptable salt, solvate, hydrate, co-crystal, clathrate, or polymorph thereof, in combination with ipilimumab, are administered in combination with a therapy conventionally used to treat or manage cancer.

Examples of such conventional therapies include, but are not limited to, surgery, chemotherapy, radiation therapy, hormonal therapy, biological therapy and immunotherapy.

In some embodiments, the methods for treating and/or managing cancer provided herein may be used in patients that have not responded to standard treatment. In one embodiment, the cancer is relapsed or refractory to conventional therapy.

In other embodiments, the methods for treating and/or managing cancer provided herein may be used in treatment naive patients, i.e., patients that have not yet received treatment.

In some embodiments, Compound A, or an enantiomer or a mixture of enantiomers thereof, or a pharmaceutically acceptable salt, solvate, hydrate, co-crystal, clathrate, or polymorph thereof, is administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with an anti-CD20 antibody, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with obinutuzumab, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with rituximab, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with an HDAC inhibitor, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with citarinostat, or a pharmaceutically acceptable salt, solvate, hydrate, co-crystal, clathrate, or polymorph thereof, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with a proteasome inhibitor, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with marizomib, or an enantiomer or a mixture of enantiomers thereof, or a pharmaceutically acceptable salt, solvate, hydrate, co-crystal, clathrate, or polymorph thereof, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with bortezomib, or an enantiomer or a mixture of enantiomers thereof, or a pharmaceutically acceptable salt, solvate, hydrate, co-crystal, clathrate, or polymorph thereof, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with carfilzomib, or an enantiomer or a mixture of enantiomers thereof, or a pharmaceutically acceptable salt, solvate, hydrate, co-crystal, clathrate, or polymorph thereof, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with ixazomib, or an enantiomer or a mixture of enantiomers thereof, or a pharmaceutically acceptable salt, solvate, hydrate, co-crystal, clathrate, or polymorph thereof, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with an anti-CD38 antibody, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with isatuximab, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with daratumumab, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with an anti-SLAMF7 antibody, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with elotuzumab, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with a nuclear export inhibitor, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with selinexor, or a geometric isomer or a mixture of geometric isomers thereof, or a pharmaceutically acceptable salt, solvate, hydrate, co-crystal, clathrate, or polymorph thereof, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with a BCL-2 inhibitor, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with venetoclax, or a pharmaceutically acceptable salt, solvate, hydrate, co-crystal, clathrate, or polymorph thereof, are administered in combination or alternation with a therapeutically effective amount of one or more additional active agents.

In some embodiments, Compound A, or an enantiomer or a mixture of enantiomers thereof, or a pharmaceutically acceptable salt, solvate, hydrate, co-crystal, clathrate, or polymorph thereof, in combination with an immune checkpoint inhibitor, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with pembrolizumab, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with nivolumab, are administered in combination or alternation with a therapeutically effective amount of one or more additional active agents. In some embodiments, Compound A, or an enantiomer or a mixture of enantiomers thereof, or a pharmaceutically acceptable salt, solvate, hydrate, co-crystal, clathrate, or polymorph thereof, in combination with ipilimumab, are administered in combination or alternation with a therapeutically effective amount of one or more additional active agents.

Additional active agents include small molecules and large molecules (e.g., proteins and antibodies), examples of which are provided herein, as well as stem cells. Methods or therapies that can be used in combination with the administration of the compounds provided herein include, but are not limited to, surgery, blood transfusions, immunotherapy, biological therapy, radiation therapy, and other non-drug based therapies presently used to treat and/or manage disease and conditions associated with or characterized by undesired angiogenesis.

In one embodiment, the additional active agent is selected from the group consisting of an alkylating agent, an adenosine analog, a glucocorticoid, a kinase inhibitor, a SYK inhibitor, a PDE3 inhibitor, a PDE7 inhibitor, doxorubicin, chlorambucil, vincristine, bendamustine, forskolin, rituximab, or a combination thereof. In one embodiment, the additional active agent is rituximab. In another embodiment, the additional active agent is prednisone.

Provided herein are methods of treating patients who have been previously treated for cancer but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

Provided herein are methods of treating patients who have been previously treated for cancer using at least two prior lines of therapy. Also provided herein are methods of treating patients who have been previously treated for cancer using at least two prior lines of therapy.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with an anti-CD20 antibody, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD20 antibody is obinutuzumab. In certain embodiments, the anti-CD20 antibody is rituximab.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with an HDAC inhibitor, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the HDAC inhibitor is citarinostat, or a pharmaceutically acceptable salt, solvate, hydrate, co-crystal, clathrate, or polymorph thereof.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with a proteasome inhibitor, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the proteasome inhibitor is marizomib, or an enantiomer or a mixture of enantiomers thereof, or a pharmaceutically acceptable salt, solvate, hydrate, co-crystal, clathrate, or polymorph thereof. In certain embodiments, the proteasome inhibitor is bortezomib, or an enantiomer or a mixture of enantiomers thereof, or a pharmaceutically acceptable salt, solvate, hydrate, co-crystal, clathrate, or polymorph thereof. In certain embodiments, the proteasome inhibitor is carfilzomib, or an enantiomer or a mixture of enantiomers thereof, or a pharmaceutically acceptable salt, solvate, hydrate, co-crystal, clathrate, or polymorph thereof. In certain embodiments, the proteasome inhibitor is ixazomib, or an enantiomer or a mixture of enantiomers thereof, or a pharmaceutically acceptable salt, solvate, hydrate, co-crystal, clathrate, or polymorph thereof.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with an anti-CD38 antibody, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-CD38 antibody is isatuximab. In certain embodiments, the anti-CD38 antibody is daratumumab.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with an anti-SLAMF7 antibody, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the anti-SLAMF7 antibody is elotuzumab.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with a nuclear export inhibitor, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the nuclear export inhibitor is selinexor, or a geometric isomer or a mixture of geometric isomers thereof, or a pharmaceutically acceptable salt, solvate, hydrate, co-crystal, clathrate, or polymorph thereof.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with a BCL-2 inhibitor, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the BCL-2 inhibitor is venetoclax, or a pharmaceutically acceptable salt, solvate, hydrate, co-crystal, clathrate, or polymorph thereof.

In certain embodiments, provided herein are methods of treating and/or managing relapsed or refractory cancer in patients, comprising administering a therapeutically effective amount of Compound A, or an enantiomer or a mixture of enantiomers thereof, or a pharmaceutically acceptable salt, solvate, hydrate, stereoisomer, tautomer or racemic mixtures thereof in combination with an immune checkpoint inhibitor, to a patient having relapsed or refractory cancer. In certain embodiment, the methods provided herein further comprise administering dexamethasone, or an enantiomer or a mixture of enantiomers thereof, or a pharmaceutically acceptable salt, solvate, hydrate, co-crystal, clathrate, or polymorph thereof. In certain embodiments, the immune checkpoint inhibitor is pembrolizumab. In certain embodiments, the immune checkpoint inhibitor is nivolumab. In certain embodiments, the immune checkpoint inhibitor is ipilimumab.

Provided herein is Compound A, or an enantiomer or a mixture of enantiomers thereof, or a pharmaceutically acceptable salt, solvate, hydrate, stereoisomer, tautomer or racemic mixtures thereof for use in such methods of treating and/or managing relapsed or refractory cancer in patients.

In certain embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.005 mg to about 1,000 mg per day, from about 0.01 mg to about 500 mg per day, from about 0.01 mg to about 250 mg per day, from about 0.01 mg to about 100 mg per day, from about 0.1 mg to about 100 mg per day, from about 0.5 mg to about 100 mg per day, from about 1 mg to about 100 mg per day, from about 0.01 mg to about 50 mg per day, from about 0.1 mg to about 50 mg per day, from about 0.5 mg to about 50 mg per day, from about 1 mg to about 50 mg per day, from about 0.02 mg to about 25 mg per day, from about 0.05 mg to about 10 mg per day, or from about 0.1 mg to about 5 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.005 mg to about 1,000 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.01 mg to about 500 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.01 mg to about 250 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.01 mg to about 100 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.1 mg to about 100 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.5 mg to about 100 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 1 mg to about 100 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.01 mg to about 50 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.1 mg to about 50 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.5 mg to about 50 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 1 mg to about 50 mg per day. In some embodiments, a therapeutically or prophylactically effective amount of Compound A is from about 0.02 mg to about 25 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.05 mg to about 10 mg per day. In some embodiments, a therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is from about 0.1 mg to about 5 mg per day.

In certain embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, about 1.5 mg, about 1.55 mg, about 1.6 mg, about 1.65 mg, about 1.7 mg, about 1.75 mg, about 1.8 mg, about 1.85 mg, about 1.9 mg, about 1.95 mg, about 2 mg, about 2.05 mg, about 2.1 mg, about 2.15 mg, about 2.2 mg, about 2.25 mg, about 2.3 mg, about 2.35 mg, about 2.4 mg, about 2.45 mg, about 2.5 mg, about 2.55 mg, about 2.6 mg, about 2.65 mg, about 2.7 mg, about 2.75 mg, about 2.8 mg, about 2.85 mg, about 2.9 mg, about 2.95 mg, about 3 mg, about 3.05 mg, about 3.1 mg, about 3.15 mg, about 3.2 mg, about 3.25 mg, about 3.3 mg, about 3.35 mg, about 3.4 mg, about 3.45 mg, about 3.5 mg, about 3.55 mg, about 3.6 mg, about 3.65 mg, about 3.7 mg, about 3.75 mg, about 3.8 mg, about 3.85 mg, about 3.9 mg, about 3.95 mg, about 4 mg, about 4.05 mg, about 4.1 mg, about 4.15 mg, about 4.2 mg, about 4.25 mg, about 4.3 mg, about 4.35 mg, about 4.4 mg, about 4.45 mg, about 4.5 mg, about 4.55 mg, about 4.6 mg, about 4.65 mg, about 4.7 mg, about 4.75 mg, about 4.8 mg, about 4.85 mg, about 4.9 mg, about 4.95, or about 5 mg per day.

In certain embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.1 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.15 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.2 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.25 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.3 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.35 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.4 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.45 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.5 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.55 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.6 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.65 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.7 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.75 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.8 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.85 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.9 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 0.95 mg per day. In some embodiments, the therapeutically or prophylactically effective amount of Compound A is or an enantiomer or a mixture of enantiomers thereof, or a pharmaceutically acceptable salt, solvate, hydrate, stereoisomer, tautomer or racemic mixtures thereof about 1 mg per day.

In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.05 mg per day. In certain embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.1 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.15 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.2 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.25 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, A is about 1.3 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.35 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.4 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.45 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.5 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.55 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.6 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.65 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.7 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.75 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.8 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.85 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.9 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 1.95 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2 mg per day.

In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.05 mg per day. In certain embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.1 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.15 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.2 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.25 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.3 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.35 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.4 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.45 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.5 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.55 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.6 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.65 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.7 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.75 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.8 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.85 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.9 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 2.95 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3 mg per day.

In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.05 mg per day. In certain embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.1 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.15 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.2 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.25 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.3 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.35 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.4 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.45 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.5 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.55 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.6 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.65 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.7 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.75 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.8 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.85 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.9 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 3.95 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4 mg per day.

In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.05 mg per day. In certain embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.1 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.15 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.2 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.25 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.3 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.35 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.4 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.45 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.5 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.55 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.6 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.65 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.7 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.75 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.8 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.85 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.9 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 4.95 mg per day. In some embodiments, the therapeutically or prophylactically effective amount of Compound A, or an enantiomer or a mixture of enantiomers thereof, or a pharmaceutically acceptable salt, solvate, hydrate, stereoisomer, tautomer or racemic mixtures thereof, is about 5 mg per day.

In one embodiment, the recommended daily dose range of Compound A, or an enantiomer or a mixture of enantiomers thereof, or a pharmaceutically acceptable salt, solvate, hydrate, co-crystal, clathrate, or polymorph thereof, for the conditions described herein lie within the range of from about 0.1 mg to about 5 mg per day, preferably given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, 1.6 mg, 1.65 mg, 1.7 mg, 1.75 mg, 1.8 mg, 1.85 mg, 1.9 mg, 1.95 mg, 2 mg, 2.05 mg, 2.1 mg, 2.15 mg, 2.2 mg, 2.25 mg, 2.3 mg, 2.35 mg, 2.4 mg, 2.45 mg, 2.5 mg, 2.55 mg, 2.6 mg, 2.65 mg, 2.7 mg, 2.75 mg, 2.8 mg, 2.85 mg, 2.9 mg, 2.95 mg, 3 mg, 3.05 mg, 3.1 mg, 3.15 mg, 3.2 mg, 3.25 mg, 3.3 mg, 3.35 mg, 3.4 mg, 3.45 mg, 3.5 mg, 3.55 mg, 3.6 mg, 3.65 mg, 3.7 mg, 3.75 mg, 3.8 mg, 3.85 mg, 3.9 mg, 3.95 mg, 4 mg, 4.05 mg, 4.1 mg, 4.15 mg, 4.2 mg, 4.25 mg, 4.3 mg, 4.35 mg, 4.4 mg, 4.45 mg, 4.5 mg, 4.55 mg, 4.6 mg, 4.65 mg, 4.7 mg, 4.75 mg, 4.8 mg, 4.85 mg, 4.9 mg, 4.95 mg, or 5 mg per day. In certain embodiments, the specific dose per day is 0.15 mg, 0.3 mg, 0.45 mg, 0.6 mg, 0.75 mg, 0.9 mg, 1 mg, 0 mg, 1.1 mg, 1.2 mg, 1.3 mg, 1.4 mg, 1.5 mg, 1.6 mg, 1.7 mg, 1.8 mg, or 1.9 mg per day.

In certain embodiments, the anti-CD20 antibody is administered in a therapeutically effective amount. In certain embodiments, the anti-CD20 antibody is obinutuzumab. In certain embodiments, obinutuzumab is administered intravenously (for example via intravenous infusion) or via subcutaneous infusion. In certain embodiments, obinutuzumab is administered in a therapeutically effective amount. In certain embodiments, obinutuzumab is administered by intravenous infusion. In certain embodiments, obinutuzumab is administered in an amount of about 1000 mg per day. In certain embodiments, obinutuzumab is administered once every 7 days, or once every 4 weeks. In certain embodiments, obinutuzumab is administered according to the locally approved label or Pharmacy manual for preparation, administration, and storage information.

In certain embodiments, the anti-CD20 is rituximab. In certain embodiments, rituximab is administered in a therapeutically effective amount. In certain embodiments, rituximab is administered intravenously. In certain embodiments, rituximab is administered in an amount of about 375 mg/m² per day. In certain embodiments, rituximab is administered by subcutaneous infusion. In certain embodiments, rituximab is administered in an amount of about 1400 mg per day. In certain embodiments, rituximab is administered once every 7 days, or once every 4 weeks. In certain embodiments, rituximab is administered according to the locally approved label or Pharmacy manual for preparation, administration, and storage information.

In certain embodiments, the HDAC inhibitor is administered in a therapeutically effective amount. In certain embodiments, the HDAC inhibitor is citarinostat, or a pharmaceutically acceptable salt, solvate, hydrate, co-crystal, clathrate, or polymorph thereof. In certain embodiments, citarinostat, or a pharmaceutically acceptable salt, solvate, hydrate, co-crystal, clathrate, or polymorph thereof, is administered according to the locally approved label or Pharmacy manual for preparation, administration, and storage information.

In certain embodiments, the proteasome inhibitor is administered in a therapeutically effective amount. In certain embodiments, the proteasome inhibitor is marizomib, or an enantiomer or a mixture of enantiomers thereof, or a pharmaceutically acceptable salt, solvate, hydrate, co-crystal, clathrate, or polymorph thereof. In certain embodiments, marizomib, or an enantiomer or a mixture of enantiomers thereof, or a pharmaceutically acceptable salt, solvate, hydrate, co-crystal, clathrate, or polymorph thereof is administered according to the locally approved label or Pharmacy manual for preparation, administration, and storage information.

In certain embodiments, the proteasome inhibitor is bortezomib, or an enantiomer or a mixture of enantiomers thereof, or a pharmaceutically acceptable salt, solvate, hydrate, co-crystal, clathrate, or polymorph thereof. In certain embodiments, bortezomib, or an enantiomer or a mixture of enantiomers thereof, or a pharmaceutically acceptable salt, solvate, hydrate, co-crystal, clathrate, or polymorph thereof, is administered in an amount of 1.3 mg/m². In certain embodiments, bortezomib, or an enantiomer or a mixture of enantiomers thereof, or a pharmaceutically acceptable salt, solvate, hydrate, co-crystal, clathrate, or polymorph thereof, is administered via a bolus intravenous injection or subcutaneous injection. In certain embodiments, bortezomib, or an enantiomer or a mixture of enantiomers thereof, or a pharmaceutically acceptable salt, solvate, hydrate, co-crystal, clathrate, or polymorph thereof, is administered once or twice weekly. In certain embodiments, bortezomib, or an enantiomer or a mixture of enantiomers thereof, or a pharmaceutically acceptable salt, solvate, hydrate, co-crystal, clathrate, or polymorph thereof, is administered according to the locally approved label or Pharmacy manual for preparation, administration, and storage information.

In certain embodiments, the proteasome inhibitor is carfilzomib, or an enantiomer or a mixture of enantiomers thereof, or a pharmaceutically acceptable salt, solvate, hydrate, co-crystal, clathrate, or polymorph thereof. In certain embodiments, carfilzomib, or an enantiomer or a mixture of enantiomers thereof, or a pharmaceutically acceptable salt, solvate, hydrate, co-crystal, clathrate, or polymorph thereof, is administered at an amount of 20/70 mg/m² once weekly, 20/56 mg/m² twice weekly, or 20/27 mg/m² twice weekly. In certain embodiments, carfilzomib, or an enantiomer or a mixture of enantiomers thereof, or a pharmaceutically acceptable salt, solvate, hydrate, co-crystal, clathrate, or polymorph thereof, is administered via intravenous infusion. In certain embodiments, carfilzomib, or an enantiomer or a mixture of enantiomers thereof, or a pharmaceutically acceptable salt, solvate, hydrate, co-crystal, clathrate, or polymorph thereof, is administered according to the locally approved label or Pharmacy manual for preparation, administration, and storage information.

In certain embodiments, the proteasome inhibitor is ixazomib, or an enantiomer or a mixture of enantiomers thereof, or a pharmaceutically acceptable salt, solvate, hydrate, co-crystal, clathrate, or polymorph thereof. In certain embodiments, ixazomib, or an enantiomer or a mixture of enantiomers thereof, or a pharmaceutically acceptable salt, solvate, hydrate, co-crystal, clathrate, or polymorph thereof, is administered at an amount of 4 mg, 3 mg, or 2.3 mg. In certain embodiments, ixazomib, or an enantiomer or a mixture of enantiomers thereof, or a pharmaceutically acceptable salt, solvate, hydrate, co-crystal, clathrate, or polymorph thereof, is administered via oral route. In certain embodiments, ixazomib, or an enantiomer or a mixture of enantiomers thereof, or a pharmaceutically acceptable salt, solvate, hydrate, co-crystal, clathrate, or polymorph thereof, is administered once a week on days 1, 8, and 15 of a 28-day treatment cycle. In certain embodiments, ixazomib, or an enantiomer or a mixture of enantiomers thereof, or a pharmaceutically acceptable salt, solvate, hydrate, co-crystal, clathrate, or polymorph thereof, is administered according to the locally approved label or Pharmacy manual for preparation, administration, and storage information.

In certain embodiments, the anti-CD38 antibody is administered in a therapeutically effective amount. In certain embodiments, the anti-CD38 antibody is isatuximab. In certain embodiments, isatuximab is administered according to the locally approved label or Pharmacy manual for preparation, administration, and storage information.

In certain embodiments, the anti-CD38 antibody is daratumumab. In certain embodiments, daratumumab is administered in an amount of 16 mg/kg actual body weight. In certain embodiments, daratumumab is administered weekly, every two weeks, every three weeks, or every four weeks. In certain embodiments, daratumumab is administered via intravenous infusion. In certain embodiments, daratumumab is administered according to the locally approved label or Pharmacy manual for preparation, administration, and storage information.

In certain embodiments, the anti-SLAMF7 antibody is administered in a therapeutically effective amount. In certain embodiments, the anti-SLAMF7 antibody is elotuzumab. In certain embodiments, elotuzumab is administered in an amount of 10 mg/kg every week for the first two 28-day cycles and every 2 weeks thereafter until disease progression or unacceptable toxicity. In certain embodiments, elotuzumab is administered intravenously. In certain embodiments, elotuzumab is administered according to the locally approved label or Pharmacy manual for preparation, administration, and storage information.

In certain embodiments, the nuclear export inhibitor is administered in a therapeutically effective amount. In certain embodiments, the nuclear export inhibitor is selinexor, or a geometric isomer or a mixture of geometric isomers thereof, or a pharmaceutically acceptable salt, solvate, hydrate, co-crystal, clathrate, or polymorph thereof. In certain embodiments, selinexor, or a geometric isomer or a mixture of geometric isomers thereof, or a pharmaceutically acceptable salt, solvate, hydrate, co-crystal, clathrate, or polymorph thereof, is administered in an amount of 80 mg on days 1 and 3 of each week. In certain embodiments, selinexor, or a geometric isomer or a mixture of geometric isomers thereof, or a pharmaceutically acceptable salt, solvate, hydrate, co-crystal, clathrate, or polymorph thereof, is administered orally. In certain embodiments, selinexor, or a geometric isomer or a mixture of geometric isomers thereof, or a pharmaceutically acceptable salt, solvate, hydrate, co-crystal, clathrate, or polymorph thereof, is administered according to the locally approved label or Pharmacy manual for preparation, administration, and storage information.

In certain embodiments, the BCL-2 inhibitor is administered in a therapeutically effective amount. In certain embodiments, the BCL-2 inhibitor is venetoclax, or a pharmaceutically acceptable salt, solvate, hydrate, co-crystal, clathrate, or polymorph thereof. In certain embodiments, venetoclax, or a pharmaceutically acceptable salt, solvate, hydrate, co-crystal, clathrate, or polymorph thereof is administered in an amount of 20 mg once daily for 7 days, followed by a weekly ramp-up dosing schedule to a daily dose of 400 mg. In certain embodiments, venetoclax, or a pharmaceutically acceptable salt, solvate, hydrate, co-crystal, clathrate, or polymorph thereof, is administered orally. In certain embodiments, venetoclax, or a pharmaceutically acceptable salt, solvate, hydrate, co-crystal, clathrate, or polymorph thereof, is administered according to the locally approved label or Pharmacy manual for preparation, administration, and storage information.

In certain embodiments, the immune checkpoint inhibitor is administered in a therapeutically effective amount. In certain embodiments, the immune checkpoint inhibitor is pembrolizumab. In certain embodiments, pembrolizumab is administered in an amount of 2 mg/kg every 3 weeks. In certain embodiments, pembrolizumab is administered via intravenous infusion. In certain embodiments, pembrolizumab is administered according to the locally approved label or Pharmacy manual for preparation, administration, and storage information.

In certain embodiments, the immune checkpoint inhibitor is nivolumab. In certain embodiments, nivolumab is administered in an amount of 3 mg/kg every 2 weeks. In certain embodiments, nivolumab is administered via intravenous infusion. In certain embodiments, nivolumab is administered according to the locally approved label or Pharmacy manual for preparation, administration, and storage information.

In certain embodiments, the immune checkpoint inhibitor is ipilimumab. In certain embodiments, ipilimumab is administered in an amount of 3 mg/kg every 3 weeks. In certain embodiments, ipilimumab is administered for a total of four doses. In certain embodiments, ipilimumab is administered via intravenous infusion. In certain embodiments, ipilimumab is administered according to the locally approved label or Pharmacy manual for preparation, administration, and storage information.

In certain embodiment, dexamethasone, or an enantiomer or a mixture of enantiomers thereof, or a pharmaceutically acceptable salt, solvate, hydrate, co-crystal, clathrate, or polymorph thereof, is administered in a therapeutically effective amount. In certain embodiments, a therapeutically or prophylactically effective amount of dexamethasone is from about 0.5 mg to about 2,000 mg per day, from about 1 mg to about 1,000 mg per day, from about 1 mg to about 500 mg per day, from about 1 mg to about 250 mg per day, from about 5 mg to about 250 mg per day, from about 7.5 mg to about 250 mg per day, from about 10 mg to about 250 mg per day, from about 20 mg to about 250 mg per day, from about 20 mg to about 200 mg per day, from about 1 mg to about 100 mg per day, from about 1 mg to about 50 mg per day, from about 0.5 mg to about 25 mg per day, or from about 0.5 mg to about 10 mg per day. In some embodiments, a therapeutically or prophylactically effective amount of dexamethasone is from about 0.5 mg to about 2,000 mg per day. In some embodiments, a therapeutically or prophylactically effective amount of dexamethasone is from about 1 mg to about 1,000 mg per day. In some embodiments, a therapeutically or prophylactically effective amount of dexamethasone is from about 1 mg to about 500 mg per day. In some embodiments, a therapeutically or prophylactically effective amount of dexamethasone is from about 1 mg to about 250 mg per day. In some embodiments, a therapeutically or prophylactically effective amount of dexamethasone is from about 5 mg to about 250 mg per day. In some embodiments, a therapeutically or prophylactically effective amount of dexamethasone is from about 7.5 mg to about 250 mg per day. In some embodiments, a therapeutically or prophylactically effective amount of dexamethasone is from about 10 mg to about 250 mg per day. In some embodiments, a therapeutically or prophylactically effective amount of dexamethasone is from about 20 mg to about 250 mg per day. In some embodiments, a therapeutically or prophylactically effective amount of dexamethasone is from about 20 mg to about 200 mg per day. In some embodiments, a therapeutically or prophylactically effective amount of dexamethasone is from about 1 mg to about 100 mg per day. In some embodiments, a therapeutically or prophylactically effective amount of dexamethasone is from about 1 mg to about 50 mg per day. In some embodiments, a therapeutically or prophylactically effective amount of dexamethasone is from about 0.5 mg to about 25 mg per day. In some embodiments, a therapeutically or prophylactically effective amount of dexamethasone is from about 0.5 mg to about 10 mg per day.

In certain embodiments, the therapeutically or prophylactically effective amount of dexamethasone is about 0.5, about 1, about 2, about 5, about 10, about 15, about 20, about 25, about 30, about 40, about 45, about 50, about 60, about 70, about 80, about 90, about 100, about 150, or about 200 mg per day. In some embodiments, the therapeutically or prophylactically effective amount of dexamethasone is about 0.5 mg per day. In some embodiments, the therapeutically or prophylactically effective amount of dexamethasone is about 1 mg per day. In some embodiments, the therapeutically or prophylactically effective amount of dexamethasone is about 2 mg per day. In some embodiments, the therapeutically or prophylactically effective amount of dexamethasone is about 5, mg per day. In some embodiments, the therapeutically or prophylactically effective amount of dexamethasone is about 10 mg per day. In some embodiments, the therapeutically or prophylactically effective amount of dexamethasone is about 15 mg per day. In some embodiments, the therapeutically or prophylactically effective amount of dexamethasone is about 20 mg per day. In some embodiments, the therapeutically or prophylactically effective amount of dexamethasone is about 25 mg per day. In some embodiments, the therapeutically or prophylactically effective amount of dexamethasone is about 30 mg per day. In some embodiments, the therapeutically or prophylactically effective amount of dexamethasone is about 40 mg per day. In some embodiments, the therapeutically or prophylactically effective amount of dexamethasone is about 45 mg per day. In some embodiments, the therapeutically or prophylactically effective amount of dexamethasone is about 50 mg per day. In some embodiments, the therapeutically or prophylactically effective amount of dexamethasone is about 60 mg per day. In some embodiments, the therapeutically or prophylactically effective amount of dexamethasone is about 70 mg per day. In some embodiments, the therapeutically or prophylactically effective amount of dexamethasone is about 80 mg per day. In some embodiments, the therapeutically or prophylactically effective amount of dexamethasone is about 90 mg per day. In some embodiments, the therapeutically or prophylactically effective amount of dexamethasone is about 100 mg per day. In some embodiments, the therapeutically or prophylactically effective amount of dexamethasone is about 150 mg per day. In some embodiments, the therapeutically or prophylactically effective amount of dexamethasone is about 200 mg per day.

In one embodiment, the recommended daily dose range of dexamethasone, or an enantiomer or a mixture of enantiomers thereof, or a pharmaceutically acceptable salt, solvate, hydrate, co-crystal, clathrate, or polymorph thereof, for the conditions described herein lie within the range of from about 0.5 mg to about 100 mg per day, preferably given as a single once-a-day dose, or in divided doses throughout a day. In some embodiments, the dosage ranges from about 1 mg to about 100 mg per day. In other embodiments, the dosage ranges from about 0.5 mg to about 20 mg per day. Specific doses include 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, or 100 mg per day.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with an anti-CD20 antibody. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with obinutuzumab. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with rituximab. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with an HDAC inhibitor. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with citarinostat, or a pharmaceutically acceptable salt, solvate, hydrate, co-crystal, clathrate, or polymorph thereof. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with a proteasome inhibitor. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with marizomib, or an enantiomer or a mixture of enantiomers thereof, or a pharmaceutically acceptable salt, solvate, hydrate, co-crystal, clathrate, or polymorph thereof. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with bortezomib, or an enantiomer or a mixture of enantiomers thereof, or a pharmaceutically acceptable salt, solvate, hydrate, co-crystal, clathrate, or polymorph thereof. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with carfilzomib, or an enantiomer or a mixture of enantiomers thereof, or a pharmaceutically acceptable salt, solvate, hydrate, co-crystal, clathrate, or polymorph thereof. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with ixazomib, or an enantiomer or a mixture of enantiomers thereof, or a pharmaceutically acceptable salt, solvate, hydrate, co-crystal, clathrate, or polymorph thereof. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with an anti-CD38 antibody. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with isatuximab. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with daratumumab. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with an anti-SLAMF7 antibody. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with elotuzumab. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with a nuclear export inhibitor. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with selinexor, or a geometric isomer or a mixture of geometric isomers thereof, or a pharmaceutically acceptable salt, solvate, hydrate, co-crystal, clathrate, or polymorph thereof. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with a BCL-2 inhibitor. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with venetoclax, or a pharmaceutically acceptable salt, solvate, hydrate, co-crystal, clathrate, or polymorph thereof. In certain embodiments, the patient to be treated with one of the methods provided herein has developed drug resistance to the anticancer therapy.

In certain embodiments, the patient to be treated with one of the methods provided herein has not been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with an immune checkpoint inhibitor. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with pembrolizumab. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with nivolumab. In certain embodiments, the patient to be treated with one of the methods provided herein has been treated with anticancer therapy prior to the administration of Compound A, or an enantiomer or a mixture of enantiomers thereof, or a pharmaceutically acceptable salt, solvate, hydrate, co-crystal, clathrate, or polymorph thereof, in combination with ipilimumab. In certain embodiments, the patient to be treated with one of the methods provided herein has developed drug resistance to the anticancer therapy.

The methods provided herein encompass treating a patient regardless of patient's age, although some diseases or disorders are more common in certain age groups. Further provided herein is a method for treating a patient who has undergone surgery in an attempt to treat the disease or condition at issue, as well in one who has not. Because the subjects with cancer have heterogeneous clinical manifestations and varying clinical outcomes, the treatment given to a particular subject may vary, depending on his/her prognosis. The skilled clinician will be able to readily determine without undue experimentation, specific secondary agents, types of surgery, and types of non-drug based standard therapy that can be effectively used to treat an individual subject with cancer.

Compound A, or an enantiomer or a mixture of enantiomers thereof, or a pharmaceutically acceptable salt, solvate, hydrate, co-crystal, clathrate, or polymorph thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Compound A, or an enantiomer or a mixture of enantiomers thereof; or a pharmaceutically acceptable salt, solvate, hydrate, co-crystal, clathrate, or polymorph thereof, may be formulated alone or together, in suitable dosage unit with pharmaceutically acceptable excipients, carriers, adjuvants and vehicles, appropriate for each route of administration.

In one embodiment, Compound A, or an enantiomer or a mixture of enantiomers thereof, or a pharmaceutically acceptable salt, solvate, hydrate, co-crystal, clathrate, or polymorph thereof, is administered orally. In another embodiment, Compound A, or an enantiomer or a mixture of enantiomers thereof; or a pharmaceutically acceptable salt, solvate, hydrate, co-crystal, clathrate, or polymorph thereof, is administered parenterally. In yet another embodiment, Compound A, or an enantiomer or a mixture of enantiomers thereof, or a pharmaceutically acceptable salt, solvate, hydrate, co-crystal, clathrate, or polymorph thereof, is administered intravenously.

Compound A, or an enantiomer or a mixture of enantiomers thereof, or a pharmaceutically acceptable salt, solvate, hydrate, co-crystal, clathrate, or polymorph thereof, can be delivered as a single dose such as, e.g., a single bolus injection, or oral tablets or pills; or over time, such as, e.g., continuous infusion over time or divided bolus doses over time. Compound A, or an enantiomer or a mixture of enantiomers thereof, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with an anti-CD20 antibody can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with obinutuzumab can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with rituximab can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with an HDAC inhibitor can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with citarinostat, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with a proteasome inhibitor can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with marizomib, or an enantiomer or a mixture of enantiomers thereof, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with bortezomib, or an enantiomer or a mixture of enantiomers thereof, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with carfilzomib, or an enantiomer or a mixture of enantiomers thereof, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with ixazomib, or an enantiomer or a mixture of enantiomers thereof, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with an anti-CD38 antibody can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with isatuximab can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with daratumumab can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with an anti-SLAMF7 antibody can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with elotuzumab can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with a nuclear export inhibitor can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with selinexor, or a geometric isomer or a mixture of geometric isomers thereof, or a pharmaceutically acceptable salt, solvate, hydrate, co-crystal, clathrate, or polymorph thereof can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with a BCL-2 inhibitor can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with venetoclax, or a pharmaceutically acceptable salt, solvate, hydrate, co-crystal, clathrate, or polymorph thereof, can be administered repeatedly if necessary, for example, until the patient experiences stable disease or regression, or until the patient experiences disease progression or unacceptable toxicity.

Compound A, or an enantiomer or a mixture of enantiomers thereof, or a pharmaceutically acceptable salt, solvate, hydrate, co-crystal, clathrate, or polymorph thereof, in combination with an immune checkpoint inhibitor can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with pembrolizumab, can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with nivolumab, can be administered repeatedly if necessary, for example, until the patient experiences stable disease or regression, or until the patient experiences disease progression or unacceptable toxicity. Compound A, or an enantiomer or a mixture of enantiomers thereof, or a pharmaceutically acceptable salt, solvate, hydrate, co-crystal, clathrate, or polymorph thereof, in combination with ipilimumab, can be administered repeatedly if necessary, for example, until the patient experiences stable disease or regression, or until the patient experiences disease progression or unacceptable toxicity.

For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MM scan and other commonly accepted evaluation modalities.

Compound A, or an enantiomer or a mixture of enantiomers thereof, or a pharmaceutically acceptable salt, solvate, hydrate, co-crystal, clathrate, or polymorph thereof, an anti-CD20 antibody, an HDAC inhibitor, a proteasome inhibitor, an anti-CD38 antibody, an anti-SLAMF7 antibody, a nuclear export inhibitor, a BCL-2 inhibitor, or an immune checkpoint inhibitor can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug).

As used herein, the term “daily” is intended to mean that a therapeutic compound, such as Compound A, is administered once or more than once each day, for example, for a period of time. The term “continuous” is intended to mean that a therapeutic compound, such as Compound A, is administered daily for an uninterrupted period of at least 10 days to 52 weeks. The term “intermittent” or “intermittently” as used herein is intended to mean stopping and starting at either regular or irregular intervals. For example, intermittent administration of Compound A is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound A, is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks.

In one embodiment, Compound A, or an enantiomer or a mixture of enantiomers thereof; or a pharmaceutically acceptable salt, solvate, hydrate, co-crystal, clathrate, or polymorph thereof, is administered once a day. In another embodiment, Compound A, or an enantiomer or a mixture of enantiomers thereof; or a pharmaceutically acceptable salt, solvate, hydrate, co-crystal, clathrate, or polymorph thereof, is administered twice a day. In yet another embodiment, Compound A, or an enantiomer or a mixture of enantiomers thereof; or a pharmaceutically acceptable salt, solvate, hydrate, co-crystal, clathrate, or polymorph thereof, is administered three times a day. In still another embodiment, Compound A, or an enantiomer or a mixture of enantiomers thereof; or a pharmaceutically acceptable salt, solvate, hydrate, co-crystal, clathrate, or polymorph thereof, is administered four times a day.

In certain embodiments, Compound A, or an enantiomer or a mixture of enantiomers thereof; or a pharmaceutically acceptable salt, solvate, hydrate, co-crystal, clathrate, or polymorph thereof, is administered once per day from one day to six months, from one week to three months, from one week to four weeks, from one week to three weeks, or from one week to two weeks. In certain embodiments, Compound A, or a pharmaceutically acceptable salt or solvate thereof, is administered once per day for one week, two weeks, three weeks, or four weeks. In one embodiment, Compound A or an enantiomer or a mixture of enantiomers thereof; or a pharmaceutically acceptable salt, solvate, hydrate, co-crystal, clathrate, or polymorph thereof, is administered once per day for one week. In another embodiment, Compound A, or an enantiomer or a mixture of enantiomers thereof; or a pharmaceutically acceptable salt, solvate, hydrate, co-crystal, clathrate, or polymorph thereof, is administered once per day for two weeks. In yet another embodiment, Compound A, or an enantiomer or a mixture of enantiomers thereof; or a pharmaceutically acceptable salt, solvate, hydrate, co-crystal, clathrate, or polymorph thereof, is administered once per day for three weeks. In still another embodiment, Compound A, or an enantiomer or a mixture of enantiomers thereof; or a pharmaceutically acceptable salt, solvate, hydrate, co-crystal, clathrate, or polymorph thereof, is administered once per day for four weeks.

In certain embodiments, Compound A, or an enantiomer or a mixture of enantiomers thereof; or a pharmaceutically acceptable salt, solvate, hydrate, co-crystal, clathrate, or polymorph thereof, is administered once per day for 21 days in each 28 day cycle, namely administered once daily for 21 days followed by 7 days of rest. In certain embodiments, Compound A, or an enantiomer or a mixture of enantiomers thereof; or a pharmaceutically acceptable salt, solvate, hydrate, co-crystal, clathrate, or polymorph thereof, is administered for one cycle. In certain embodiments, Compound A, or an enantiomer or a mixture of enantiomers thereof; or a pharmaceutically acceptable salt, solvate, hydrate, co-crystal, clathrate, or polymorph thereof, is administered for two cycles. In certain embodiments, Compound A, or an enantiomer or a mixture of enantiomers thereof; or a pharmaceutically acceptable salt, solvate, hydrate, co-crystal, clathrate, or polymorph thereof, is administered for three cycles. In certain embodiments, Compound A, or an enantiomer or a mixture of enantiomers thereof; or a pharmaceutically acceptable salt, solvate, hydrate, co-crystal, clathrate, or polymorph thereof, is administered for four cycles. In certain embodiments, Compound A, or an enantiomer or a mixture of enantiomers thereof; or a pharmaceutically acceptable salt, solvate, hydrate, co-crystal, clathrate, or polymorph thereof, is administered for seven or more cycles.

In certain embodiments, Compound A, or an enantiomer or a mixture of enantiomers thereof; or a pharmaceutically acceptable salt, solvate, hydrate, co-crystal, clathrate, or polymorph thereof, is administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more additional agents. In certain embodiments, the additional agent is selected from the group consisting of an anti-CD20 antibody, an HDAC inhibitor, a proteasome inhibitor, an anti-CD38 antibody, an anti-SLAMF7 antibody, a nuclear export inhibitor, a BCL-2 inhibitor, and an immune checkpoint inhibitor. In certain embodiments, Compound A, or an enantiomer or a mixture of enantiomers thereof; or a pharmaceutically acceptable salt, solvate, hydrate, co-crystal, clathrate, or polymorph thereof, in combination with the one or more additional agents can also be administered on an alternating dosing schedule, with or without a resting period (e.g., no therapeutic agent is administered on certain days of the schedule). In certain embodiments, the administration of Compound A, or an enantiomer or a mixture of enantiomers thereof; or a pharmaceutically acceptable salt, solvate, hydrate, co-crystal, clathrate, or polymorph thereof, in combination with the one or more additional agents includes, but is not limited to, sequential administration and concomitant administration.

Also provided herein is Compound A for use in any of the methods provided herein. Also provided herein is Compound A in combination with a second agent provided herein for use in any of the methods provided herein.

### Pharmaceutical Compositions and Dosage Forms

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) an anti-CD20 antibody. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) obinutuzumab. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) rituximab. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) an HDAC inhibitor. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) citarinostat, or a pharmaceutically acceptable salt, solvate, hydrate, co-crystal, clathrate, or polymorph thereof. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) a proteasome inhibitor. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) marizomib, or an enantiomer or a mixture of enantiomers thereof, or a pharmaceutically acceptable salt, solvate, hydrate, co-crystal, clathrate, or polymorph thereof. In another embodiment, pharmaceutical compositions and dosage forms further comprise one or more excipients. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) bortezomib, or an enantiomer or a mixture of enantiomers thereof, or a pharmaceutically acceptable salt, solvate, hydrate, co-crystal, clathrate, or polymorph thereof. In another embodiment, pharmaceutical compositions and dosage forms further comprise one or more excipients. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) carfilzomib, or an enantiomer or a mixture of enantiomers thereof, or a pharmaceutically acceptable salt, solvate, hydrate, co-crystal, clathrate, or polymorph thereof. In another embodiment, pharmaceutical compositions and dosage forms further comprise one or more excipients. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) ixazomib, or an enantiomer or a mixture of enantiomers thereof, or a pharmaceutically acceptable salt, solvate, hydrate, co-crystal, clathrate, or polymorph thereof. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) an anti-CD38 antibody. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) isatuximab. In another embodiment, pharmaceutical compositions and dosage forms further comprise one or more excipients. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) daratumumab. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) an anti-SLAMF7 antibody. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) elotuzumab. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) a nuclear export inhibitor. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) selinexor, or a geometric isomer or a mixture of geometric isomers thereof, or a pharmaceutically acceptable salt, solvate, hydrate, co-crystal, clathrate, or polymorph thereof. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) a BCL-2 inhibitor. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) venetoclax, or a pharmaceutically acceptable salt, solvate, hydrate, co-crystal, clathrate, or polymorph thereof. In another embodiment, pharmaceutical compositions and dosage forms further comprise one or more excipients.

In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) an immune checkpoint inhibitor. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) pembrolizumab. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) nivolumab. In one embodiment, provided herein are pharmaceutical compositions and dosage forms, which comprise (i) Compound A, or an enantiomer or a mixture of enantiomers thereof, or a pharmaceutically acceptable salt, solvate, hydrate, co-crystal, clathrate, or polymorph thereof, in combination with (ii) ipilimumab. In another embodiment, pharmaceutical compositions and dosage forms further comprise one or more excipients.

In certain embodiments, pharmaceutical compositions and dosage forms provided herein also comprise one or more additional active agents in amounts effective for achieving a modulation of disease or disease symptoms, including those described herein. Examples of optional additional active agents are provided herein (see, e.g., definitions section).

In certain embodiments, the pharmaceutical compositions provided herein may be administered orally, parenterally, by inhalation spray, topically, rectally, nasally, buccally, vaginally or via an implanted reservoir, preferably by oral administration or administration by injection. Oral delivery formats include, but are not limited to, tablets, capsules, caplets, solutions, suspensions, and syrups, and may also comprise a plurality of granules, beads, powders or pellets that may or may not be encapsulated. In one embodiment, the pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In certain embodiments, dosage forms provided herein for Compound A, or an enantiomer or a mixture of enantiomers thereof, or a pharmaceutically acceptable salt, solvate, hydrate, co-crystal, clathrate, or polymorph thereof, are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

In one embodiment, obinutuzumab is formulated as described in the package insert for GAZYVA®. In one embodiment, rituximab is formulated as described in the package insert for RITUXAN™ (an anti-CD20 antibody sold under the trademark RITUXAN). In one embodiment, citarinostat, or a pharmaceutically acceptable salt, solvate, hydrate, co-crystal, clathrate, or polymorph thereof, is formulated as described in published clinical trial protocols. In one embodiment, marizomib, or an enantiomer or a mixture of enantiomers thereof, or a pharmaceutically acceptable salt, solvate, hydrate, co-crystal, clathrate, or polymorph thereof, is formulated as described in published clinical trial protocols. In one embodiment, bortezomib, or an enantiomer or a mixture of enantiomers thereof, or a pharmaceutically acceptable salt, solvate, hydrate, co-crystal, clathrate, or polymorph thereof, is formulated as described in the package insert for VALCADE®. In one embodiment, carfilzomib, or an enantiomer or a mixture of enantiomers thereof, or a pharmaceutically acceptable salt, solvate, hydrate, co-crystal, clathrate, or polymorph thereof, is formulated as described in the package insert for KYPROLIS®. In one embodiment, ixazomib, or an enantiomer or a mixture of enantiomers thereof, or a pharmaceutically acceptable salt, solvate, hydrate, co-crystal, clathrate, or polymorph thereof, is formulated as described in the package insert for NINLARO®. In one embodiment, isatuximab is formulated as described in published clinical trial protocols. In one embodiment, daratumumab is formulated as described in the package insert for DARZALEX®. In one embodiment, elotuzumab is formulated as described in the package insert for EMPLICITI™ (a SLAMF7-directed immunostimulatory antibody sold under the trademark of EMPLICITI). In one embodiment, selinexor, or a geometric isomer or a mixture of geometric isomers thereof, or a pharmaceutically acceptable salt, solvate, hydrate, co-crystal, clathrate, or polymorph thereof, is formulated as described in the package insert for XPOVIO™ (a nuclear export inhibitor sold under the trademark XPOVIO). In one embodiment, venetoclax, or a pharmaceutically acceptable salt, solvate, hydrate, co-crystal, clathrate, or polymorph thereof, is formulated as described in the package insert for VENCLEXTA™ (a BCL-2 inhibitor sold under the trademark VENCLEXTA). In one embodiment, pembrolizumab is formulated as described in the package insert for KEYTRUDA®. In one embodiment, nivolumab is formulated as described in the package insert for OPDIVO®. In one embodiment, nivolumab is formulated as described in the package insert for YERVOY™ (a human cytotoxic T-lymphocyte antigen 4 (CTLA-4)-blocking antibody sold under the trademark YERVOY).

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed herein are pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions provided herein can comprise excipients that are listed, for example, in the U.S. Pharmacopeia (USP) 25-NF20 (2002). In certain embodiments, lactose-free compositions comprise active ingredients, a binder/filler, and a lubricant in pharmaceutically compatible and pharmaceutically acceptable amounts. In certain embodiments, lactose-free dosage forms comprise active ingredients, microcrystalline cellulose, pre-gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in certain embodiments, provided herein are anhydrous compositions packaged using materials to prevent exposure to water such that they can be included in suitable formulary kits. Examples of suitable packaging include, but are not limited to, hermetically sealed foils, plastics, unit dose containers (e.g., vials), blister packs, and strip packs.

Encompassed herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

1. Oral Dosage Forms

In certain embodiments, pharmaceutical compositions provided herein that are suitable for oral administration are formulated as discrete dosage forms, examples of which include, but are not limited to, tablets (e.g., chewable tablets), caplets, capsules, and liquids (e.g., flavored syrups). Such dosage forms contain predetermined amounts of active ingredients and may be prepared by some known methods of pharmacy. See generally, Remington's Pharmaceutical Sciences, 18th ed., Mack Publishing, Easton Pa. (1990).

In certain embodiments, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms may be prepared by some known methods of pharmacy. In certain embodiments, pharmaceutical compositions and dosage forms are prepared by uniformly and intimately admixing the active ingredients with liquid carriers, finely divided solid carriers, or both, and then shaping the product into the desired presentation if necessary.

In certain embodiments, a tablet is prepared by compression or molding. In certain embodiments, compressed tablets are be prepared by compressing in a suitable machine the active ingredients in a free-flowing form, e.g., powder or granules, optionally mixed with an excipient. In certain embodiments, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, AVICEL-PH-101, AVICEL-PH-103 AVICEL RC-581, AVICEL-PH-105 (FMC Corporation, American Viscose Division, Avicel Sales, Marcus Hook, Pa.), and mixtures thereof. An specific binder is a mixture of microcrystalline cellulose and sodium carboxymethyl cellulose (e.g., AVICEL RC-581). Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation. In certain embodiments, the pharmaceutical compositions provided herein comprise from about 0.5 to about 15 weight percent or from about 1 to about 5 weight percent of disintegrant.

Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200, W.R. Grace Co., Baltimore, Md.), a coagulated aerosol of synthetic silica (Degussa Co. of Plano, Tex.), CAB-O-SIL (a pyrogenic silicon dioxide, Cabot Co. of Boston, Mass.), and mixtures thereof. In certain embodiments, if used at all, lubricants are used in an amount of less than about 1 weight percent of the pharmaceutical compositions or dosage forms into which they are incorporated.

In certain embodiments, provided herein is a solid oral dosage form, comprising Compound A, or an enantiomer or a mixture of enantiomers thereof, or a pharmaceutically acceptable salt, solvate, hydrate, co-crystal, clathrate, or polymorph thereof; and one or more excipients selected from anhydrous lactose, microcrystalline cellulose, polyvinylpyrrolidone, stearic acid, colloidal anhydrous silica, and gelatin.

In certain embodiments, provided herein is a solid oral dosage form, comprising Compound A, or an enantiomer or a mixture of enantiomers thereof, or a pharmaceutically acceptable salt, solvate, hydrate, co-crystal, clathrate, or polymorph thereof; and anhydrous lactose, microcrystalline cellulose, polyvinylpyrrolidone, stearic acid, colloidal anhydrous silica, and gelatin.

In certain embodiments, provided herein is a solid oral dosage form, comprising a hydrochloride salt of Compound A, or an enantiomer or a mixture of enantiomers thereof, or a pharmaceutically solvate, hydrate, co-crystal, clathrate, or polymorph thereof; and one or more excipients selected from anhydrous lactose, microcrystalline cellulose, polyvinylpyrrolidone, stearic acid, colloidal anhydrous silica, and gelatin.

In certain embodiments, provided herein is a solid oral dosage form, comprising a hydrochloride salt of Compound A, or an enantiomer or a mixture of enantiomers thereof, or a pharmaceutically solvate, hydrate, co-crystal, clathrate, or polymorph thereof; and anhydrous lactose, microcrystalline cellulose, polyvinylpyrrolidone, stearic acid, colloidal anhydrous silica, and gelatin.

2. Delayed Release Dosage Forms

In certain embodiments, the active ingredients provided herein are administered by controlled release means or by delivery devices. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated herein by reference in its entirety. In certain embodiments, such dosage forms are be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Some suitable vehicles that can be used to provide parenteral dosage forms provided herein include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients provided herein can also be incorporated into the parenteral dosage forms provided herein. For example, cyclodextrin and its derivatives can be used to increase the solubility of Compound A, or an enantiomer or a mixture of enantiomers thereof, or a pharmaceutically acceptable salt, solvate, hydrate, co-crystal, clathrate, or polymorph thereof. See, e.g., U.S. Pat. No. 5,134,127, which is incorporated herein by reference in its entirety.

4. Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein depend on the particular tissue to which a given pharmaceutical composition or dosage form will be applied. With that fact in mind, in certain embodiments, the excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Additional examples of such ingredients can be found, e.g., in Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5. Kits

In certain embodiments, active ingredients provided herein are not administered to a patient at the same time or by the same route of administration. Therefore, encompassed herein are kits which, when used by the medical practitioner, can simplify the administration of appropriate amounts of active ingredients to a patient.

In certain embodiments, a kit provided herein comprises a dosage form of a Compound A, or an enantiomer or a mixture of enantiomers thereof, or a pharmaceutically acceptable salt, solvate, hydrate, co-crystal, clathrate, or polymorph thereof. In certain embodiments, the kits provided herein further comprise an anti-CD20 antibody, an HDAC inhibitor, a proteasome inhibitor, an anti-CD38 antibody, an anti-SLAMF7 antibody, a nuclear export inhibitor, a BCL-2 inhibitor, and/or an immune checkpoint inhibitor. In certain embodiments, the kits provided herein further comprise obinutuzumab, rituximab, citarinostat or a pharmaceutically acceptable salt, solvate, hydrate, co-crystal, clathrate, or polymorph thereof, marizomib or an enantiomer or a mixture of enantiomers thereof, or a pharmaceutically acceptable salt, solvate, hydrate, co-crystal, clathrate, or polymorph thereof, bortezomib or an enantiomer or a mixture of enantiomers thereof, or a pharmaceutically acceptable salt, solvate, hydrate, co-crystal, clathrate, or polymorph thereof, carfilzomib or an enantiomer or a mixture of enantiomers thereof, or a pharmaceutically acceptable salt, solvate, hydrate, co-crystal, clathrate, or polymorph thereof, ixazomib or an enantiomer or a mixture of enantiomers thereof, or a pharmaceutically acceptable salt, solvate, hydrate, co-crystal, clathrate, or polymorph thereof, isatuximab, daratumumab, elotuzumab, selinexor or a geometric isomer or a mixture of geometric isomers thereof, or a pharmaceutically acceptable salt, solvate, hydrate, co-crystal, clathrate, or polymorph thereof, venetoclax or a pharmaceutically acceptable salt, solvate, hydrate, co-crystal, clathrate, or polymorph thereof, pembrolizumab, nivolumab, and/or ipilimumab. In certain embodiments, the kit provided herein further comprises additional active ingredient(s) include, but are not limited to, those provided herein. In certain embodiment, the kits provided herein further comprise dexamethasone, or an enantiomer or a mixture of enantiomers thereof, or a pharmaceutically acceptable salt, solvate, hydrate, co-crystal, clathrate, or polymorph thereof.

In certain embodiments, the kit provided herein further comprises a device that is used to administer the active ingredients. Examples of such devices include, but are not limited to, syringes, drip bags, patches, and inhalers.

In certain embodiments, the kit provided herein further comprises cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

Certain embodiments of the invention are illustrated by the following non-limiting examples.

### Example 1: A Phase 1, Multicenter, Open-Label Study to Assess Safety, Pharmacokinetics, and Preliminary Efficacy of Compound A, Alone and in Combination with Rituximab or Obinutuzumab in Subjects with Relapsed or Refractory Non-Hodgkin Lymphomas (R/R NHL) and Classical Hodgkin Lymphoma (cHL)

### I. Study Objectives

The primary objectives of the study are to determine the safety and tolerability of Compound A alone and in combination with rituximab or obinutuzumab in subjects with R/R NHL and cHL, and to define the maximum tolerated dose (MTD) and/or the recommended phase 2 dose (RP2D) of Compound A in subjects with R/R NHL and cHL.

The secondary objectives are to characterize the pharmacokinetic (PK) of Compound A alone and in combination with rituximab or obinutuzumab and to evaluate the preliminary efficacy of Compound A alone and in combination with rituximab or obinutuzumab in R/R NHL and cHL.

The exploratory objectives are to correlate PK with safety profile, clinical activity and pharmacodynamic (PD) biomarkers, to explore PD biomarkers of Compound A activity, and to evaluate effect of Compound A on normal T, B and NK cells in peripheral blood.

Study Endpoints is shown in Table 1.

TABLE 1
Study Endpoints.

| Endpoint | Name | Description | Timeframe |
|---|---|---|---|
| Primary | Safety and tolerability | Adverse events (AEs) | From first dose to 28 |
| | | evaluated using NCI | days after last dose of |
| | | CTCAE criteria, v.5.0, | study treatment, or |
| | | including treatment- | indefinitely for SAEs |
| | | emergent adverse events | occurring after 28 |
| | | (TEAEs), laboratory | days from last dose of |
| | | assessments, vital signs, | study treatment that |
| | | ECOG performance status | are suspected to be |
| | | and ECG results. | related to study |
| | | | treatment |
| | MTD and RP2D | Frequency of DLTs (cf. | DLT evaluation period |
| | | definition) to define MTD | defined from first |
| | | and establish the RP2D of | Compound A dose |
| | | Compound A as | through completion of |
| | | monotherapy and in | C1 |
| | | combination with rituximab | |
| | | or obinutuzumab | |
| Secondary | PK | Cₘₐₓ, C_{through}, AUC, tₘₐₓ, t_{1/2}, | C1D8, C2D1 and |
| | | CL/F, V/F for Compound A | C3D1 |
| | Efficacy (Part B) | Best overall response rate | Every 3 cycles during |
| | | (ORR) | treatment period and |
| | | Complete response rate | then every 6 months. |
| | | (CRR) | |
| | | Time to response (TTR) | |
| | | Duration of response (DOR) | |
| | | Progression-free survival | |
| | | (PFS) | |
| | | Overall survival (OS) | |
| | | According to 2014 Lugano | |
| | | classification | |
| Exploratory | PK correlation | To explore the relationship | C1D8, C2D1 and |
| | | between systemic exposure | C3D1 |
| | | of Compound A, PD | |
| | | biomarkers, and clinical | |
| | | activity. | |
| | PD biomarkers | Assess changes in | Screening, C1D8 |
| | | mechanism of action | |
| | | markers in paired fine | |
| | | needle aspirates of tumor | |
| | | tissue | |
| | PD biomarker | Explore perturbations in | Archival/S creening, |
| | | pathway expression and | C1D15, EOT |
| | | immune cell populations in | |
| | | paired tumor samples | |
| | PD biomarker | Assess changes in tumor | Screening, C2D1, |
| | | cell clonality | C3D1, C5D1, EOT |
| | PD biomarkers | Evaluate the | C1D1 |
| | | pharmacodynamic (PD) | |
| | | effects of Compound A on | |
| | | degradation of Aiolos in | |
| | | peripheral B and T-cells | |
| | PD biomarkers | Explore changes in | Screening, C1D1, |
| | | peripheral immune cell | C1D15, C2D1, C2D15 |
| | | populations by | |
| | | immunophenotyping | |

NCI CTCAE: National Cancer Institute Common Terminology Criteria for Adverse Events.
ECOG: Eastern Cooperative Oncology Group.
ECG: electrocardiogram

### II. Study Design

This is a Phase 1, multicohort, multicenter study to demonstrate safety, tolerability and preliminary signal of efficacy of Compound A given alone or in combination with rituximab or obinutuzumab in subjects with R/R NHL or cHL.

A dose finding (DF) phase with 3 parallel cohorts (DF-A cohort: monotherapy, DF-B cohort: combination with rituximab, DF-C cohort: combination with obinutuzumab) is followed by a dose confirmation (DC) phase with 5 cohorts (DC-A: MCL, Compound A monotherapy; DC-B: PTCL, Compound A monotherapy; DC-C: cHL, Compound A monotherapy; DC-D: aggressive B-cell lymphoma, in combination with rituximab; DC-E: aggressive B-cell lymphoma, in combination with obinutuzumab; DC-F: FL and MZL in combination with obinutuzumab).

A. Dose Finding Phase (DF)

DF consists of a dose-finding phase with study treatment given for 21 of 28-day cycles using an mTPI-2 design with target toxicity level (TTL) 0.2. Dose limiting toxicity (DLT) is assessed to determine MTD during the first treatment cycle in each cohort.

A DLT is defined as below:

Hematologic DLT
Any episode of febrile neutropenia;
Grade 4 neutropenia lasting ≥7 days;
Grade 4 thrombocytopenia lasting ≥7 days;
Grade 3 thrombocytopenia with clinically significant bleeding and/or requiring platelet transfusion;
Grade 4 anemia, not explained by underlying disease Non-hematologic DLT
Any non-hematological toxicity ≥Grade 3 except for
   Grade 3-4 Infusion related reaction related to rituximab or obinutuzumab that resolves to ≤grade 2 within 72 hours after initiation of maximal medical intervention
   Grade 3-4 tumor lysis syndrome assessed using Cairo-Bishop grading system that resolves to ≤grade 2 within 72 hours after initiation of maximal medical intervention
   Any grade 3-4 event related to disease progression, including flare effect
Any treatment interruption greater than 2 weeks due to adverse event

All subjects within the same dose-level cohort are treated and observed for at least 28 days (C1) after the first dose of Compound A. In case of a DL is declared tolerated, DL+1 is opened. In case of DL1 is declared non-tolerated, subjects are enrolled in DL-1 (and eventually DL-2). The dose levels being studied are shown in Table 2. Beyond C1, dose reduction is permitted according to safety evaluation at each visit (see below). Dose reductions are presented on Table 3.

In the monotherapy arm (cohort DF-A), subjects with R/R NHL or cHL receive oral Compound A at dose specified by cohort dose level from D1-21 of each 28-day cycle.

In the combination treatment arm (cohort DF-B), subjects with R/R B-cell NHL receive oral Compound A at dose specified by cohort dose level from D1-21 of each 28-day cycle, and rituximab is administered at a dose of 375 mg/m² IV at C1D1 and then either by SC infusion at a dose of 1400 mg on D8, 15 and 22 of C1 and then every 28-day cycle at D1 from C2 to 5 or by IV infusion at a dose of 375 mg/m² according to the same schedule.

In the combination treatment arm (cohort DF-C), subjects with R/R FL (grade 1-3a), MZL or aggressive NHL receive oral Compound A at dose specified by cohort dose level from D1-21 of each 28-day cycle, and obinutuzumab as IV infusion at 1000 mg every week in C1 (D1, 8, 15) and on D1 of every 28-day cycle from C2 to 6.

Dose escalation on monotherapy (cohort DF-A) and combination (cohorts DF-B and DF-C) may occur in parallel with evaluation of the recommended 21 out of 28-day dose. The study treatment is continued until disease progression, unacceptable toxicity, consent withdrawal by subject or physician decision, except for FL and MZL patients where Compound A is administered up to 12 cycles.

Approximately 66 subjects (22 subjects in each cohort) are treated and evaluated in DF phase for MTD; however, the total number of subjects in DF phase depends on the number of dose levels needed to establish the MTD.

B. Dose Confirmation (DC) Phase

Once MTD is established for cohort DF-A, DF-B or DF-C, the DC phase of the study is initiated.

A total of 20 subjects are enrolled at MTD in each cohort:

DC-A: R/R MCL, Compound A monotherapy;

DC-B: R/R PTCL, Compound A monotherapy;

DC-C: R/R cHL, Compound A monotherapy;

DC-D: R/R aggressive B-cell lymphoma, in combination with rituximab;

DC-E: R/R aggressive B-cell lymphoma, in combination with obinutuzumab;

DC-F: R/R FL and MZL in combination with obinutuzumab.

In DC phase, up to 120 subjects (20 subjects per DC cohort) are enrolled to further evaluate safety and evaluation of preliminary efficacy.

The RP2D is decided as a dose level of the MTD determination or as a lower dose level of the MTD dose level determination. This decision is based upon the safety +/−PK data.

TABLE 2
Dose finding phase: cohorts and dose-level assignments.

| | | | Cohort DF-B | Cohort DF-C |
|---|---|---|---|---|
| | | Cohort DF-A | Compound A + | Compound A + |
| | Dose-level | Compound A | rituximab | obinutuzumab |
| DF | −2 | 0.6 mg QD | 0.6 mg QD | 0.6 mg QD |
| phase | −1 | 0.75 mg QD | 0.75 mg QD | 0.75 mg QD |
| | 1 | 1.0 mg QD | 1.0 mg QD | 1.0 mg QD |
| | 2 | 1.3 mg QD | 1.3 mg QD | 1.3 mg QD |
| | 3 | 1.6 mg QD | 1.6 mg QD | 1.6 mg QD |
| | 4 | 1.9 mg QD | 1.9 mg QD | 1.9 mg QD |

TABLE 3
dose-level reductions.

| Starting Dose | Part 1 | | |
|---|---|---|---|
| Level | DLR-1 | DLR-2 | DLR-3 |
| 0.6 mg | 0.45 mg | 0.3 mg | 0.15 mg |
| 0.75 mg | 0.6 mg | 0.45 mg | 0.3 mg |
| 1.0 mg | 0.75 mg | 0.6 mg | 0.45 mg |
| 1.3 mg | 1 mg | 0.75 mg | 0.6 mg |
| 1.6 mg | 1.3 mg | 1.0 mg | 0.75 mg |
| 1.9 mg | 1.6 mg | 1.3 mg | 1.0 mg |

### III. Study Population/Estimated Number of Patients

It is estimated that up to 166 subjects are enrolled in the study, including approximately 66 subjects (22 subjects per cohort) in the dose finding phase, and up to 120 subjects (20 per dose confirmation cohort) in the dose confirmation phase.

### IV. Key Inclusion Criteria

Subjects must satisfy the following criteria to be enrolled in the study:
1. Subject is ≥18 years of age the time of signing the informed consent form (ICF).
2. Subject must understand and voluntarily sign an ICF prior to any study-related assessments/procedures being conducted.
3. Subject is willing and able to adhere to the study visit schedule and other protocol requirements.
4. Subject has histologically confirmed (per local evaluation) diagnosis of lymphoma according to 2016 WHO classification including:
   a. Aggressive B-cell lymphoma including DLBCL not otherwise specified (NOS), High-grade B-cell lymphoma, with MYC and BCL2 and/or BCL6 rearrangements, grade 3b follicular lymphoma and primary mediastinal large B-cell lymphoma. In DC phase, subjects are enrolled in DC-D or DC-E according to physician decision.
   b. FL grade 1-3a. In DC phase, subjects are enrolled in DC-F.
   c. MZL including extranodal marginal zone lymphoma (ENMZL) of mucosa-associated lymphoid tissue (MALT lymphoma), nodal marginal zone lymphoma (NMZL) and splenic marginal zone lymphoma (SMZL). In DC phase, subjects are enrolled in DC-F.
   d. MCL (non blastoid). In DC phase, subjects are enrolled in DC-A.
   e. Peripheral T-cell lymphoma (PTCL) including PTCL NOS, AITL and others PTCL with T-follicular helper (TFH) phenotype, ALK-anaplastic large-cell lymphoma (ALCL). In DC phase, subjects are enrolled in DC-B.
   f. cHL. In DC phase, subjects are enrolled in DC-C.
5. Relapsed or refractory disease according to the following definitions:
   a. Aggressive B-cell lymphoma: after at least two prior lines of therapy including R-CHOP-like regimen OR after one prior line of standard therapy and being not eligible for autologous stem cell transplantation (ASCT). Subjects previously treated with CAR-T therapy can be enrolled.
   b. FL and MZL: following at least 2 prior lines of systemic therapy (including at least one anti-CD20 containing and one alkylating-containing regimen) and in need for treatment. For SMZL, splenectomy is considered as one line. For ENMZL, Helicobacter pylori eradication is not considered as a previous line.
   c. MCL: following at least two prior lines of therapy including at least one immunochemotherapy and one BTK inhibitor.
   d. PTCL: following at least two prior systemic lines of therapy.
   e. cHL: following at least two prior systemic lines of therapy, including brentuximab vedotin and anti-PD1.
6. Subjects must have measurable disease defined by at least one FDG-avid lesion for FDG-avid subtype and one bi-dimensionally measurable (>1.5 cm in longest diameter) disease by computed tomography (CT) or magnetic resonance imaging (MM), as defined by the Lugano classification (Cheson et al., J. Clin. Oncol. 2014, 32(27):3059-3068).
7. Subject has an Eastern Cooperative Oncology Group (ECOG) performance status of 0, 1 or 2.
8. Subjects must have the following laboratory values:
   a. Absolute neutrophil count (ANC)≥1.5×10⁹/L or ≥1.0×10⁹/L in case of documented bone marrow involvement (>50% or tumor cells), without growth factor support for 7 days (14 days if pegfilgastrim);
   b. Hemoglobin (Hb)≥8 g/dL;
   c. Platelets (Plt)≥75×10⁹/L or ≥50×10⁹/L in case of documented bone marrow involvement (>50% or tumor cells), without transfusion for 7 days;
   d. Aspartate aminotransferase/serum glutamic oxaloacetic transaminase (AST/SGOT) and alanine aminotransferase/serum glutamate pyruvic transaminase (ALT/SGPT)≤2.5× upper limit of normal (ULN);
   e. Serum total bilirubin ≤2.0 mg/dL (34 μmol/L) except in cases of Gilbert syndrome, then ≤5.0 mg/dl (86 μmol/L);
   f. Estimated serum creatinine clearance of ≥50 mL/min using the Cockcroft-Gault equation, MDRD or CKD-EPI formula or directly determined from the 24-hour urine collection method.
9. Females of childbearing potential (FCBP):
   a. Either commit to true abstinence from heterosexual contact (which must be reviewed on a monthly basis and source documented) or agree to use, and be able to comply with, at least 2 effective contraceptive methods (oral, injectable, or implantable hormonal contraceptive; tubal ligation; intra-uterine device; barrier contraceptive with spermicide; or vasectomized partner), one of which must be barrier, from signing the ICF, at least 28 days before starting Compound A, throughout the study, and for up to 28 days following the last dose of Compound A and up to one year following the last dose of rituximab; and
   b. Have 2 negative pregnancy tests as verified by the Investigator prior to starting Compound A:
      i. a negative serum pregnancy test (sensitivity of at least 25 mIU/mL) at Screening (between 10 to 14 days prior to C1 D1);
      ii. a negative serum or urine pregnancy test (Investigator's discretion) within 24 hours prior to C1 D1 of study treatment (note that the screening serum pregnancy test can be used as the test prior to D1 study treatment if it is performed within the prior 24 hours).
   c. Avoid conceiving for 28 days after the last dose of Compound A.
   d. Agree to ongoing pregnancy testing during the course of the study, and after the end of study treatment. This applies even if the subject practices true abstinence (True abstinence is acceptable when this is in line with the preferred and usual lifestyle of the subject. In contrast, periodic abstinence (eg, calendar, ovulation, symptothermal, post-ovulation methods) and withdrawal are not acceptable methods of contraception) from heterosexual contact.
   e. Agree to refrain from donating ova while on Compound A for 30 days after its discontinuation.
   f. Agree to abstain from breastfeeding or providing breast milk while on Compound A and for 28 days after its discontinuation.
10. Males must
   a. practice true abstinence (True abstinence is acceptable when this is in line with the preferred and usual lifestyle of the subject. In contrast, periodic abstinence (eg, calendar, ovulation, symptothermal, post-ovulation methods) and withdrawal are not acceptable methods of contraception) (which must be reviewed on a monthly basis) or agree to use a condom (a latex condom is recommended) during sexual contact with a pregnant female or a FCBP and avoid conceiving from the date of signing the ICF, while participating in the study, during dose interruptions, and for at least 90 days following Compound A discontinuation, even if he has undergone a successful vasectomy.
   b. agree to refrain from donating semen or sperm while on Compound A and for 90 days after its discontinuation.

### V. Key Exclusion Criteria

The presence of any of the following excludes a subject from enrollment:
1. Subject has any significant medical condition, laboratory abnormality, or psychiatric illness that prevents the subject from participating in the study.
2. Subject has any condition including the presence of laboratory abnormalities, which places the subject at unacceptable risk if he/she were to participate in the study.
3. Subject has any condition that confounds the ability to interpret data from the study.
4. Subject has life expectancy ≤2 months.
5. Subjects who have aggressive lymphoma relapse requiring immediate cytoreductive therapy to avoid potential life-threatening consequences (e.g. due to tumor location).
6. Subject with prior grade 3-4 infusion related reaction with rituximab (for Compound A+rituximab cohorts) or obinutuzumab (for Compound A+obinutuzumab cohorts).
7. Subject has received prior systemic anti-cancer treatment, including CAR-T or any T-cell targeting treatment (approved or investigational)≤5 half-lives or 4 weeks prior to starting Compound A, whichever is shorter.
8. Subject has received prior therapy with Cereblon-modulating drug (e.g. lenalidomide)≤4 weeks prior to starting Compound A.
9. Subject is a pregnant or breastfeeding female or intends to become pregnant during participation in the study.
10. Subject has documented or suspected CNS involvement of disease.
11. Persistent diarrhea or malabsorption ≥grade 2 NCI-CTCAE v5.0, despite medical management.
12. Peripheral neuropathy ≥NCI CTCAE grade 2.
13. Subject is on chronic systemic immunosuppressive therapy or corticosteroids (e.g. prednisone or equivalent not to exceed 10 mg per day within the last 14 days). Stable use of inhaled or topical corticosteroids is allowed.
14. Subject has impaired cardiac function or clinically significant cardiac diseases, including any of the following:
   a. Left ventricular ejection fraction (LVEF)<45% as determined by multigated acquisition scan (MUGA) or echocardiogram (ECHO);
   b. Complete left bundle branch or bifascicular block;
   c. Congenital long QT syndrome;
   d. Persistent or clinically meaningful ventricular arrhythmias;
   e. QTcF ≥470 msec on screening electrocardiogram (ECG; mean of triplicate recordings);
   f. Unstable angina pectoris or myocardial infarction ≤3 months prior to starting.
15. Subject had prior autologous SCT ≤3 months prior to starting Compound A and any treatment-related toxicity is unresolved (grade >1).
16. Subject had prior allogeneic SCT with either standard or reduced intensity conditioning ≤6 months prior to starting Compound A and any treatment-related toxicity is unresolved (grade >1) or with clinically significant graft-versus-host disease (GVHD). The use of topical steroids for ongoing skin or ocular GVHD is permitted.
17. Subject had major surgery ≤2 weeks prior to starting Compound A. Subjects must have recovered from any clinically significant effects of recent surgery.
18. Prior radiotherapy within one month prior to starting study drug.
19. Known seropositive for or active viral infection with human immunodeficiency virus (HIV).
20. Subject has known chronic active hepatitis B (HBs Ag positive and/or anti-HBc positive with viral DNA positive) or C (positive serology requiring treatment and/or with evidence of liver damage) virus (HBV/HCV) infection.
21. Subject has a history of concurrent second cancers requiring active, ongoing systemic treatment.
22. Concurrent administration of strong CYP3A4/5 modulators (see corresponding section).
23. Subjects with gastrointestinal disease that may significantly alter the absorption of Compound A.
24. Subject is unable or unwilling to undergo protocol required thromboembolism prophylaxis.

### VI. Length of Study

The study consists of a screening and a treatment phases for subjects in both DF and DC phases.

The screening phase of this study may not exceed a 28-day window prior to the start of IP (C1 D1).

The treatment phase consists of 28-day cycles for all cohorts. Treatment at each dose level and in each cohort of the study continues until progression, unacceptable toxicity, consent withdrawal by subjects or according to physician decision, except for patients with FL or MZL who receive up to 12 cycles. There is an end of treatment (EOT) visit to collect safety and efficacy assessments.

All subjects have long-term follow-up. Subjects are evaluated every 6 months for 5 years and then annually. Subjects are followed for second primary malignancy (SPM) at least for 5 years after the last subject enrolled in the study.

### VII. Overview of Key Efficacy Assessments

Physical examination including ECOG performance status.

Complete blood cell count.

CT-scan and/or PET-CT scan.

Bone marrow evaluation: biopsy, aspiration.

For additional detail please refer to the table of events.

### VIII. Overview of Key Safety Assessments

Complete physical examination including vital signs and venous thromboembolism (VTE) monitoring.

Clinical laboratory evaluations (hematology, serum chemistry, urinalysis).

Pregnancy testing/counseling.

Electrocardiogram (ECG).

Concomitant medications and procedures.

Adverse events (AEs).

Second primary malignancies (SPMs).

For additional detail please refer to the table of events.

### IX. Study Treatments

A. Compound A

Compound A is formulated capsules and labeled appropriately as investigational product for this study.

VTE prophylaxis is recommended and is given according to physician decision.

Dosing interruptions and reductions are permitted throughout the study. Subjects are evaluated for adverse events at each visit with the NCI CTCAE (v 5.0) used as a guide for the grading of severity.

Instructions for Compound A dose interruptions and reductions are provided in Table 3 and Table 4.

To initiate a new cycle of Compound A following a dose interruption, the absolute neutrophil count must be ≥1,000/μL with or without granulocyte colony-stimulating factor (G-CSF) (not permitted during C1), the platelet count must be ≥50,000/μL, and non-hematologic AEs must be grade 0 or 1 or improved as outlined in Table 4.

If recovery from toxicities is prolonged and Compound A dose withholding is beyond 14 days, then the dose of Compound A should be decreased by one dose level when dosing is resumed in the new cycle.

No dose re-escalation is permitted for Compound A.

TABLE 4
guidelines for dose interruptions and reductions.

| | |
|---|---|
| Toxicity | Dose Reduction |

Hematologic Toxicity

| | |
|---|---|
| Neutropenia. | Stop the dose for the remainder of the Compound A |
| Grade 4 neutropenia | treatment cycle. If the subject is not receiving G-CSF |
| (ANC <500/μL) | therapy for given cycle (not permitted during C1), initiate |
| or febrile neutropenia | G-CSF therapy. On D1 of the next cycle, the dose of |
| (fever ≥38.5° C. and | Compound A may be maintained if neutropenia was the |
| ANC <1,000/μL). | only Compound A-related toxicity requiring a dose |
| | modification and G-CSF treatment is continued. ANC |
| | must return to ≥1,000/μL to resume dosing. |
| Grade 4 thrombocytopenia (platelet | Stop the dose for the remainder of the Compound A |
| count <25,000/μL) or grade 3 | treatment cycle. Decrease by one dose level when |
| thrombocytopenia with bleeding or | restarting treatment when platelet count returns to |
| any requirement for a platelet | ≥50,000/L. |
| transfusion. | |

Non-Hematologic Toxicity

| | |
|---|---|
| Grade 3 rash. | Withhold dose of Compound A for remainder of cycle. |
| | Decrease by one dose level Compound A when treatment |
| | is restarted (rash must be resolved or improved to ≤ grade |
| | 1 before dose resumption). |
| Grade 4 rash or blistering. | Permanently discontinue IP. |
| Grade 3-4 thrombosis or embolism. | Stop the dose of Compound A for remainder of cycle. |
| | Anti coagulation therapy should be adapted based on the |
| | clinical and investigational results. |
| | Decrease by one dose level of Compound A when |
| | restarting treatment. |
| Grade 3 peripheral neuropathy. | Stop the dose of Compound A for remainder of cycle. |
| | Decrease Compound A by one dose level when restarting |
| | treatment (neuropathy must resolve to ≤ grade 1). |
| Toxicity | Dose Reduction |

Hematologic Toxicity

| | |
|---|---|
| Grade 4 peripheral neuropathy. | Discontinue the subject from IP. |

Other Non-Hematologic Toxicity

| | |
|---|---|
| Other ≥ grade 3 Compound A-related | Withhold dose for remainder of cycle. Decrease by one |
| AE | dose level when dosing resumed at next cycle (adverse |
| | event must be resolved or improved to ≤ grade 2 before |
| | restarting dosing). |

B. Rituximab

Rituximab is in formulated IV and SC infusion and labeled appropriately as investigational product for this study.

Depending on local authorization and physician preference, protocol allows the use of: Rituximab IV, including generic and Rituximab SC.

Dosing schedule and dose adjustments are to follow current version of Prescribing Information and local guidelines. Prophylaxis before infusion is given according to physician and current version of Prescribing Information.

C. Obinutuzumab

Obinutuzumab is in formulated IV infusion and labeled appropriately as investigational product for this study.

Dosing schedule and dose adjustments are to follow current version of Prescribing Information. Prophylaxis before infusion is given according to physician and current version of Prescribing Information.

### X. Statistical Methods

This phase 1 study consists of a dose finding and a dose confirmation phase.

Primary endpoint is defined in the corresponding section.

In the dose finding phase, a Modified Toxicity Probability Interval-2 (mTPI-2) design (Ji, 2010; Ji, 2013; Guo, 2017) is used to determine the MTD/RP2D. Approximately up to 22 subjects are enrolled in each cohort of the dose finding phase. The number of subjects depend on the number of dose levels being tested (based on the occurrence of DLT) and may exceed these approximations.

The target toxicity rate for the MTD is 0.2. Subjects are enrolled in cohorts of size ≥4 with maximum sample size of 10 for each dose level. The initial dose level of Compound A is be 1.0 mg. Subsequent dose levels are determined by the SRC based on safety data from the study.

Dose escalation/de-escalation is according to a reliable extension of the modified toxicity probability interval (Ji, 2010; Ji, 2013; Guo, 2017) with prior Beta (0.5,0.5) and is decided according to the rule displayed in Table 5.

TABLE 5
Rules for Dose Escalation/de-escalation.

| | Number of subjects treated at the current dose | | | | | | | |
|---|---|---|---|---|---|---|---|---|
| Action | 3 | 4 | 5 | 6 | 7 | 8 | 9 | 10 |
| Escalate if number | 0 | 0 | 1 | 1 | 1 | 1 | 1 | 1 |
| of subjects who | | | | | | | | |
| experienced DLT≤ | | | | | | | | |
| De-escalate if number | 1 | 2 | 2 | 2 | 2 | 3 | 3 | 3 |
| of subjects who | | | | | | | | |
| experienced DLT≥ | | | | | | | | |
| Eliminate if number | 3 | 3 | 3 | 3 | 4 | 4 | 4 | 5 |
| of subjects who | | | | | | | | |
| experienced DLTs≥ | | | | | | | | |

DLT = dose-limiting toxicity.

In the decision rule, “eliminate” means that the current and higher doses are eliminated from the trial to prevent treating any future subjects at these doses because they are overly toxic. When the dose is eliminated, the dose is automatically de-escalated to the next lower level. If none of the actions (i.e., escalation, de-escalation or elimination) is triggered, next cohort is treated at the current dose.

The MTD is selected as the tested dose for which the estimate of the toxicity rate is closest to the target toxicity rate of 0.2. If there are ties, the higher dose level is selected when the estimate is lower than the target toxicity rate; and the lower dose level is selected when the estimate is greater than the target toxicity rate.

For the dose confirmation phase, up to approximately 20 subjects are enrolled in each cohort to evaluate safety, PK/PD, and preliminary efficacy.

### Example 2: Evaluation of Anti-Proliferative Activity

The anti-proliferative activity of pomalidomide (treatment of cells with 0.001-1.0 μM) combined with bortezomib (62.5 and 125.0 pM) or Compound A-S (treatment of cells with 0.1-10.0 nM) combined with bortezomib (62.5 and 125.0 pM) was measured by 3H-thymidine incorporation in MM1.S cells after 3 days of treatment in each condition. Combination indices (CI) were calculated using the Chou-Talalay Method for each combination. CI values below 1 indicated synergistic activity of combination.

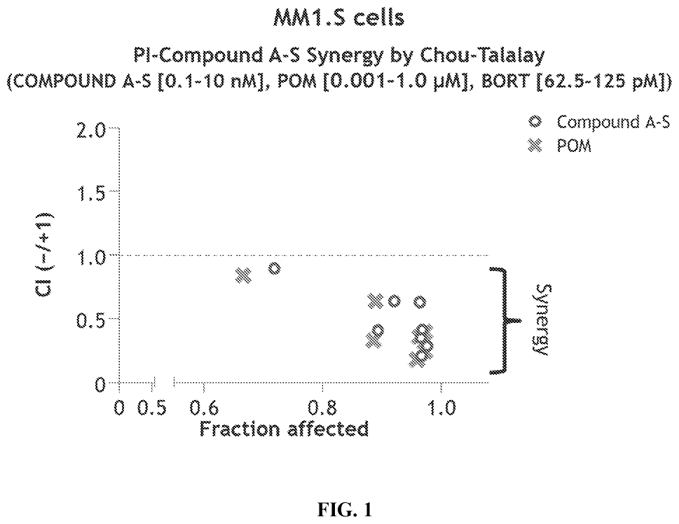

As shown in FIG. 1, pomalidomide showed synergistic anti-proliferative activity in combination with bortezomib. Compound A-S showed synergistic anti-proliferative activity in combination with bortezomib (FIG. 1).

### Example 3: Evaluation of the Apoptosis

Apoptosis was measured in KMS-12BM cells by flow cytometry using Annexin-V (AnnV+; x-axis) and ToPro3+ (y-axis) staining after either vehicle (DMSO), pomalidomide (POM) or Compound A-S treatment over 3 days following 1 hour pulse treatment with DMSO, Bortezomib (BORT) or Carfilzomib (CFZ) at indicated concentrations.

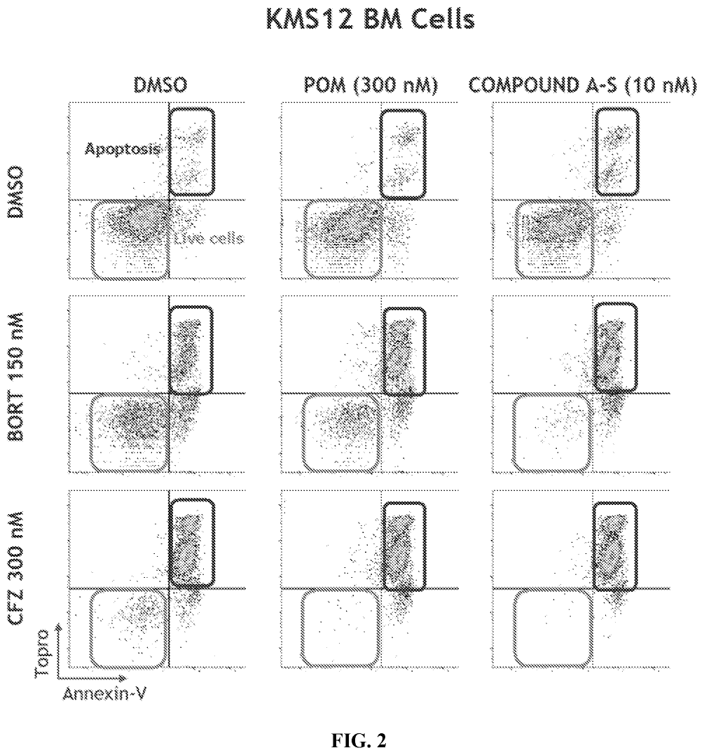

As shown in FIG. 2, pomalidomide induced cell killing in combination with proteasome inhibitors, bortezomib or carfilzomib. Compound A-S induced cell killing in combination with proteasome inhibitors, bortezomib or carfilzomib (FIG. 2).

The examples set forth above are provided to give those of ordinary skill in the art with a complete description of how to make and use the claimed embodiments, and are not intended to limit the scope of what is provided herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating or managing cancer, comprising administering to a patient in need of such treatment or management an amount of from about 0.01 mg to about 5 mg per day of a compound, wherein the compound is Compound A 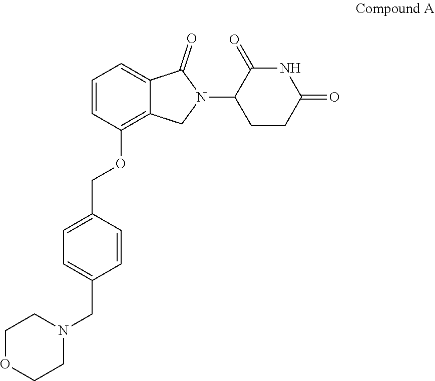 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

2. A method of treating or managing cancer, comprising administering to a patient in need of such treatment or management (i) a therapeutically effective amount of a compound in combination with (ii) an anti-CD20 antibody, wherein the compound is Compound A 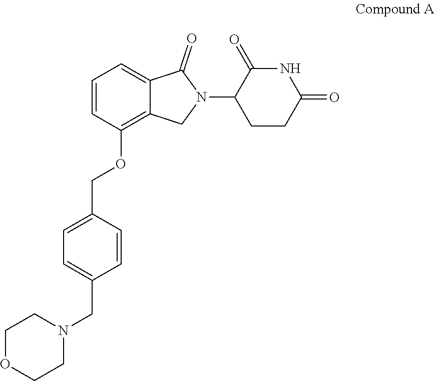 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

3. A method of treating or managing cancer, comprising administering to a patient in need of such treatment or management (i) a therapeutically effective amount of a compound in combination with (ii) an histone deacetylase (HDAC) inhibitor, wherein the compound is Compound A 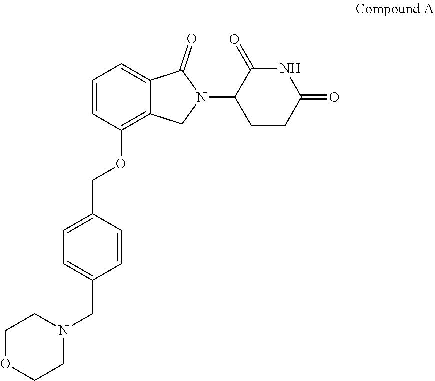 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

4. A method of treating or managing cancer, comprising administering to a patient in need of such treatment or management (i) a therapeutically effective amount of a compound in combination with (ii) a proteasome inhibitor, wherein the compound is Compound A 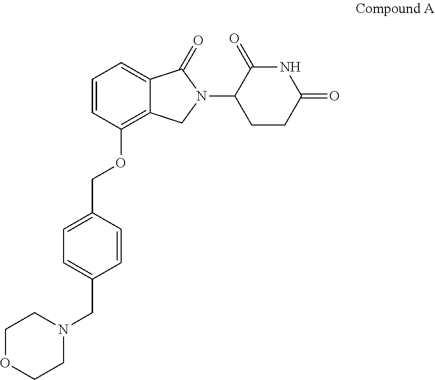 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

5. A method of treating or managing cancer, comprising administering to a patient in need of such treatment or management (i) a therapeutically effective amount of a compound in combination with (ii) an anti-CD38 antibody, wherein the compound is Compound A 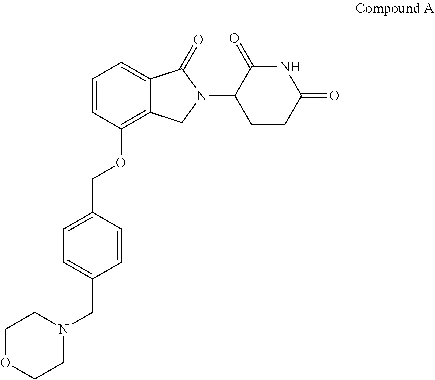 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

6. A method of treating or managing cancer, comprising administering to a patient in need of such treatment or management (i) a therapeutically effective amount of a compound in combination with (ii) an anti-SLAMF7 antibody, wherein the compound is Compound A 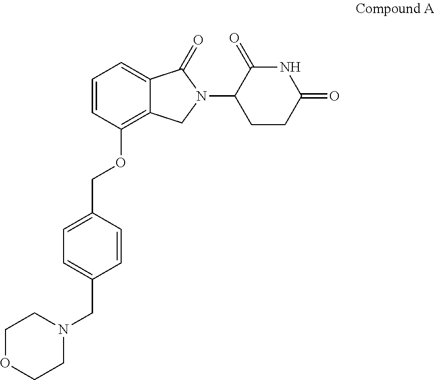 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

7. A method of treating or managing cancer, comprising administering to a patient in need of such treatment or management (i) a therapeutically effective amount of a compound in combination with (ii) a nuclear export inhibitor, wherein the compound is Compound A 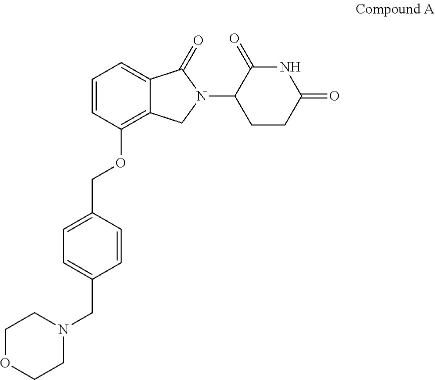 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

8. A method of treating or managing cancer, comprising administering to a patient in need of such treatment or management (i) a therapeutically effective amount of a compound in combination with (ii) a BCL-2 inhibitor, wherein the compound is Compound A 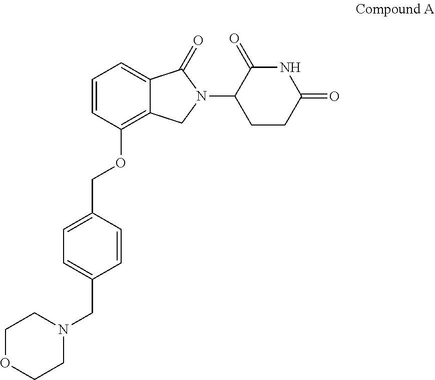 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

9. A method of treating or managing cancer, comprising administering to a patient in need of such treatment or management (i) a therapeutically effective amount of a compound in combination with (ii) an immune checkpoint inhibitor, wherein the compound is Compound A 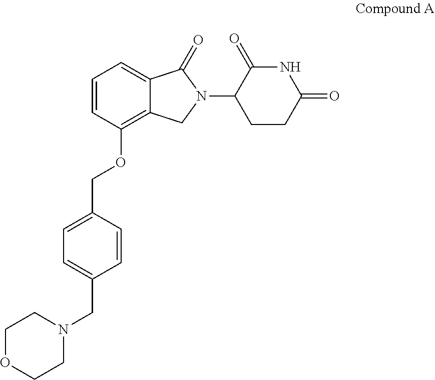 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

10. The method of any one of claims 1 to 9, wherein the cancer is non-Hodgkin lymphoma (NHL).

11. The method of claim 10, wherein the NHL is diffuse large B-cell lymphoma (DLBCL), follicular lymphoma (FL), marginal zone lymphoma (MZL), mantle cell lymphoma (MCL), peripheral T-cell lymphoma (PTCL), or primary central nervous system lymphoma (PCNSL).

12. The method of claim 11, wherein the NHL is DLBCL.

13. The method of claim 11, wherein the NHL is FL.

14. The method of claim 11, wherein the NHL is MZL.

15. The method of claim 11, wherein the NHL is MCL.

16. The method of claim 11, wherein the NHL is PTCL.

17. The method of claim 11, wherein the NHL is PCNSL.

18. The method of claim 10, wherein the NHL is relapsed or refractory NHL.

19. The method of claim 18, wherein the NHL is relapsed or refractory DLBCL.

20. The method of claim 18, wherein the NHL is relapsed or refractory FL.

21. The method of claim 18, wherein the NHL is relapsed or refractory MZL.

22. The method of claim 18, wherein the NHL is relapsed or refractory MCL.

23. The method of claim 18, wherein the NHL is relapsed or refractory PTCL.

24. The method of claim 18, wherein the NHL is relapsed or refractory PCNSL.

25. The method of any one of claims 18 to 24, wherein the subject has failed at least one prior therapy.

26. The method of any one of claims 1 to 17, wherein the NHL is newly diagnosed.

27. The method of any one of claims 1 to 9, wherein the cancer is Hodgkin Lymphoma (HL).

28. The method of claim 27, wherein the HL is classical Hodgkin Lymphoma (cHL).

29. The method of any one of claims 27 to 28, wherein the HL is relapsed or refractory HL.

30. The method of claim 29, wherein the HL is relapsed or refractory cHL.

31. The method of any one of claims 29 to 30, wherein the subject has failed at least one prior therapy.

32. The method of any one of claims 27 to 28, wherein the HL is newly diagnosed.

33. The method of any one of claims 1 to 32, wherein the compound is administered orally.

34. The method of any one of claims 1 to 33, wherein the compound is administered in an amount of about 0.1 mg, 0.15 mg, 0.2 mg, 0.3 mg, 0.4 mg, 0.45 mg, 0.5 mg, 0.6 mg, 0.7 mg, 0.75 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg or 1.9 mg per day.

35. The method of any one of claims 1 to 34, wherein the compound is administered once daily for 21 days followed by 7 days of rest.

36. The method of any one of claims 2 and 10 to 35, wherein the anti-CD20 antibody is obinutuzumab.

37. The method of claim 36, wherein obinutuzumab is administered by intravenous infusion.

38. The method of any one of claims 36 to 37, wherein obinutuzumab is administered in an amount of about 1000 mg per day.

39. The method of any one of claims 36 to 38, wherein obinutuzumab is administered once every 7 days, or once every 4 weeks.

40. The method of any one of claims 2 and 10 to 35, wherein the anti-CD20 antibody is rituximab.

41. The method of claim 40, wherein rituximab is administered intravenously.

42. The method of any one of claims 40 to 41, wherein rituximab is administered in an amount of about 375 mg/m² per day.

43. The method of any one of claims 40 to 42, wherein rituximab is administered by subcutaneous infusion.

44. The method of any one of claims 40 to 43, wherein rituximab is administered in an amount of about 1400 mg per day.

45. The method of any one of claims 40 to 44, wherein rituximab is administered once every 7 days, or once every 4 weeks.

46. The method of any one of claims 3 and 10 to 35, wherein the HDAC inhibitor is citarinostat (ACY-241), or a pharmaceutically acceptable salt, solvate, hydrate, co-crystal, clathrate, or polymorph thereof.

47. The method of any one of claims 4 and 10 to 35, wherein the proteasome inhibitor is marizomib (salinosporamide A), or an enantiomer or a mixture of enantiomers thereof, or a pharmaceutically acceptable salt, solvate, hydrate, co-crystal, clathrate, or polymorph thereof.

48. The method of any one of claims 4 and 10 to 35, wherein the proteasome inhibitor is bortezomib, or an enantiomer or a mixture of enantiomers thereof, or a pharmaceutically acceptable salt, solvate, hydrate, co-crystal, clathrate, or polymorph thereof.

49. The method of any one of claims 4 and 10 to 35, wherein the proteasome inhibitor is carfilzomib, or an enantiomer or a mixture of enantiomers thereof, or a pharmaceutically acceptable salt, solvate, hydrate, co-crystal, clathrate, or polymorph thereof.

50. The method of any one of claims 4 and 10 to 35, wherein the proteasome inhibitor is ixazomib, or an enantiomer or a mixture of enantiomers thereof, or a pharmaceutically acceptable salt, solvate, hydrate, co-crystal, clathrate, or polymorph thereof.

51. The method of any one of claims 5 and 10 to 35, wherein the anti-CD38 antibody is isatuximab.

52. The method of any one of claims 5 and 10 to 35, wherein the anti-CD38 antibody is daratumumab.

53. The method of any one of claims 6 and 10 to 35, wherein the anti-SLAMF7 antibody is elotuzumab.

54. The method of any one of claims 7 and 10 to 35, wherein the nuclear export inhibitor is selinexor, or a geometric isomer or a mixture of geometric isomers thereof, or a pharmaceutically acceptable salt, solvate, hydrate, co-crystal, clathrate, or polymorph thereof.

55. The method of any one of claims 8 and 10 to 35, wherein the BCL-2 inhibitor is venetoclax, or a pharmaceutically acceptable salt, solvate, hydrate, co-crystal, clathrate, or polymorph thereof.

56. The method of any one of claims 9 to 35, wherein the immune checkpoint inhibitor is pembrolizumab.

57. The method of any one of claims 9 to 35, wherein the immune checkpoint inhibitor is nivolumab.

58. The method of any one of claims 9 to 35, wherein the immune checkpoint inhibitor is ipilimumab.

59. The method of any one of claims 1 to 58, wherein the method further comprises administering dexamethasone, or an enantiomer or a mixture of enantiomers thereof, or a pharmaceutically acceptable salt, solvate, hydrate, co-crystal, clathrate, or polymorph thereof.

60. The method of any one of claims 1 to 59, wherein the compound is Compound A-S 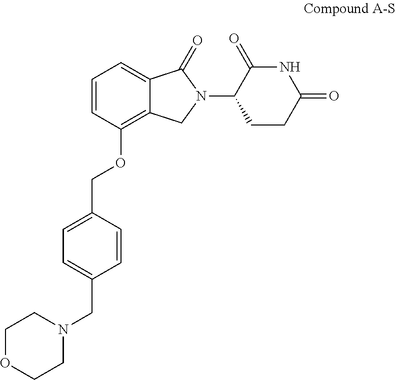 [SMILES: O=C1CC[C@H](N2Cc3c(OCc4ccc(CN5CCOCC5)cc4)cccc3C2=O)C(=O)N1]
or a pharmaceutically acceptable salt, solvate, hydrate, co-crystal, clathrate, or polymorph thereof.

61. The method of claim 60, wherein the compound is hydrochloride salt of Compound A-S.

62. A compound for use in a method of treating or managing cancer, wherein the method comprises administering to a patient in need of such treatment or management an amount of from about 0.01 mg to about 5 mg per day of the compound, wherein the compound is Compound A 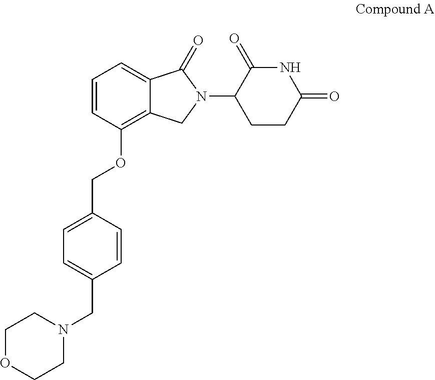 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof.

63. A compound for use in a method of treating or managing cancer, wherein the method comprises administering to a patient in need of such treatment or management (i) a therapeutically effective amount of the compound in combination with (ii) a second agent, wherein the compound is Compound A 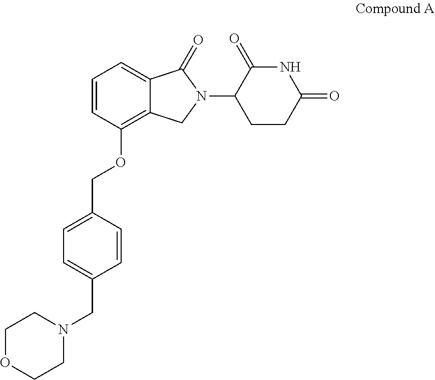 [SMILES: O=C1CCC(N2Cc3c(OCc4ccc(CN5CCOCC5)cc4)cccc3C2=O)C(=O)N1]
or an enantiomer or mixture of enantiomers thereof, or a pharmaceutically acceptable salt, solvate, hydrate, co-crystal, clathrate, or polymorph thereof, and
wherein the second agent is an anti-CD20 antibody, an histone deacetylase (HDAC) inhibitor, a proteasome inhibitor, an anti-CD38 antibody, an anti-SLAMF7 antibody, a nuclear export inhibitor, a BCL-2 inhibitor, or an immune checkpoint inhibitor.

64. The compound for use of claim 62 or 63, wherein the cancer is non-Hodgkin lymphoma (NHL).

65. The compound for use of claim 64, wherein the NHL is diffuse large B-cell lymphoma (DLBCL), follicular lymphoma (FL), marginal zone lymphoma (MZL), mantle cell lymphoma (MCL), peripheral T-cell lymphoma (PTCL), or primary central nervous system lymphoma (PCNSL).

66. The compound for use of claim 64, wherein the NHL is relapsed or refractory NHL, optionally wherein the NHL is relapsed or refractory DLBCL, relapsed or refractory FL, relapsed or refractory MZL, relapsed or refractory MCL, relapsed or refractory PTCL, or relapsed or refractory PCNSL.

67. The compound for use of claim 66, wherein the subject has failed at least one prior therapy.

68. The compound for use of claim 62 or 63, wherein the cancer is Hodgkin Lymphoma (HL), wherein optionally the HL is classical Hodgkin Lymphoma (cHL), and/or wherein the HL is relapsed or refractory HL, wherein optionally the HL is relapsed or refractory cHL, and/or wherein the subject has failed at least one prior therapy.

69. The compound for use of any one of claim 62 to 65 or 68, wherein the NHL or HL is newly diagnosed.

70. The compound for use of any one of claims 62 to 69, wherein the compound is administered in an amount of about 0.1 mg, 0.15 mg, 0.2 mg, 0.3 mg, 0.4 mg, 0.45 mg, 0.5 mg, 0.6 mg, 0.7 mg, 0.75 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg or 1.9 mg per day, and/or wherein the compound is administered once daily for 21 days followed by 7 days of rest.

71. The compound for use of any one of claims 63 to 70, wherein the anti-CD20 antibody is obinutuzumab or rituximab.

72. The compound for use of claim 71, wherein a) obinutuzumab is administered by intravenous infusion, and/or wherein obinutuzumab is administered in an amount of about 1000 mg per day, and/or wherein obinutuzumab is administered once every 7 days, or once every 4 weeks, or
b) rituximab is administered intravenously, and/or wherein rituximab is administered in an amount of about 375 mg/m² per day, and/or wherein rituximab is administered by subcutaneous infusion, and/or wherein rituximab is administered in an amount of about 1400 mg per day, and/or wherein rituximab is administered once every 7 days, or once every 4 weeks.

73. The compound for use of any one of claims 63 to 70, wherein the HDAC inhibitor is citarinostat (ACY-241), or a pharmaceutically acceptable salt, solvate, hydrate, co-crystal, clathrate, or polymorph thereof, or
wherein the proteasome inhibitor is marizomib (salinosporamide A), or an enantiomer or a mixture of enantiomers thereof, or a pharmaceutically acceptable salt, solvate, hydrate, co-crystal, clathrate, or polymorph thereof, or
wherein the proteasome inhibitor is bortezomib, or an enantiomer or a mixture of enantiomers thereof, or a pharmaceutically acceptable salt, solvate, hydrate, co-crystal, clathrate, or polymorph thereof, or
wherein the proteasome inhibitor is carfilzomib, or an enantiomer or a mixture of enantiomers thereof, or a pharmaceutically acceptable salt, solvate, hydrate, co-crystal, clathrate, or polymorph thereof, or
wherein the proteasome inhibitor is ixazomib, or an enantiomer or a mixture of enantiomers thereof, or a pharmaceutically acceptable salt, solvate, hydrate, co-crystal, clathrate, or polymorph thereof, or
wherein the anti-CD38 antibody is isatuximab, or
wherein the anti-CD38 antibody is daratumumab, or
wherein the anti-SLAMF7 antibody is elotuzumab, or
wherein the nuclear export inhibitor is selinexor, or a geometric isomer or a mixture of geometric isomers thereof, or a pharmaceutically acceptable salt, solvate, hydrate, co-crystal, clathrate, or polymorph thereof, or
wherein the BCL-2 inhibitor is venetoclax, or a pharmaceutically acceptable salt, solvate, hydrate, co-crystal, clathrate, or polymorph thereof.

74. The compound for use of any one of claims 63 to 70, wherein the immune checkpoint inhibitor is pembrolizumab, nivolumab, or ipilimumab.

75. The compound for use of any one of claims 62 to 74, wherein the method further comprises administering dexamethasone, or an enantiomer or a mixture of enantiomers thereof, or a pharmaceutically acceptable salt, solvate, hydrate, co-crystal, clathrate, or polymorph thereof.

76. The compound for use of any one of claims 62 to 75, wherein the compound is Compound A-S 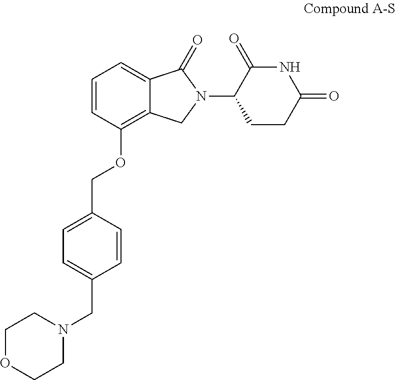 [SMILES: O=C1CC[C@H](N2Cc3c(OCc4ccc(CN5CCOCC5)cc4)cccc3C2=O)C(=O)N1]
or a pharmaceutically acceptable salt, solvate, hydrate, co-crystal, clathrate, or polymorph thereof, wherein optionally the compound is hydrochloride salt of Compound A-S.
